(19) United States
(12) Patent Application Publication
(10) Pub. No.: US 2023/0167476 A1
(43) Pub. Date: Jun. 1, 2023

(54) CHIMERIC POLYPEPTIDES AND METHODS OF PREPARING SAME

(71) Applicant: RAMOT AT TEL-AVIV UNIVERSITY LTD., Tel Aviv (IL)

(72) Inventors: Tamir TULLER, Tel Aviv (IL); Matan ARBEL, Tel Aviv (IL); Itamar MENUHIN, Ramat Gan (IL); Itai KATZIR, Tel Aviv (IL); Shaked BERGMAN, Tel Aviv (IL); Doron NAKI, Ramat Gan (IL)

(21) Appl. No.: 17/921,035

(22) PCT Filed: Apr. 22, 2021
(86) PCT No.: PCT/IL2021/050464

§ 371 Date: Oct. 24, 2022

Related U.S. Application Data
(60) Provisional application No. 63/013,665, filed on Apr. 22, 2020.

(51) Int. Cl. C12P 21/00; G16B 40/00; C12N 15/81

(52) U.S. Cl. CPC C12P 21/00; G16B 40/00; C12N 15/81

(57) ABSTRACT

The present invention is directed to a chimeric polypeptide including a polypeptide of interest N-terminal to an essential protein of a target cell. Further provided is a polynucleotide encoding the chimeric polypeptide, a method of producing thereof, and a method for producing a protein of interest. The chimeric polypeptide of the invention improves the genetic stability of the polypeptide of interest ensuring its expression over time.

### CROSS-REFERENCE TO RELATED APPLICATIONS

This application claims the benefit of priority of U.S. Provisional Patent Application No. 63/013,665, titled “CHIMERIC POLYPEPTIDES AND METHODS OF PREPARING SAME”, filed Apr. 22, 2020, the contents of which are incorporated herein by reference in their entirety.

### FIELD OF THE INVENTION

The present invention relates to synthetic biology, including evolutionary stable genetic circuits.

### BACKGROUND

Advances in synthetic biology have led to an arsenal of proof-of-principle bacterial circuits that can be leveraged for applications ranging from therapeutics to bioproduction. A unifying challenge for most applications is the presence of strong selective pressure that will lead to an unstable evolutionary genetic construct that will undeniably cease to work in a short period of time. This predicament is hindering any major advances in biosynthetic engineering, or more importantly, its implementation.

Any foreign gene (hence referred to as target gene) expressed in high levels in any microorganism for therapeutics or bioproduction purposes will cause a huge metabolic load on its host, compelling the organism to manufacture numerous proteins against its own benefit thus greatly decreasing its fitness. This target gene, whether manufactured solely, or as part of a genetic circuit, will inevitably undergo a random mutation which will either greatly reduce or erase altogether the expression of the target protein. The specific organism that underwent this mutation will now possess a far greater fitness compared to its companions (as it is not carrying the metabolic burden of the genetic circuit) and will inevitably take over the population, thus erasing the hard efforts to obtain the genetic circuit. Currently, there is no reasonable solution to this well characterized problem. Previous endeavors have shown many faults and problems. Either the solution is highly specific and requires a great deal of preliminary work, time and investment, making it not economic, or the solution is limited to very controlled and specific environments, thus greatly limiting the possibilist of biosynthetic engineering.

An essential protein is any protein that is critical for the vitality of the organism in which it is active. For example, in the genetic model organism, the baker yeast Saccharomyces cerevisiae, there are about 1,200 such proteins, out of a total of about 6,000 genes. Almost all mutations in an essential gene will in turn lead to the fatality of the organism, whose genome underwent the mutation.

In the field of biosynthetic biology there is still a great need for a system and a method for generating genetic circuits which will enable increased protein production capacities while maintaining genetic and evolutionary stability and reducing manufacturing cost.

### SUMMARY

According to a first aspect, there is provided a composition comprising a plurality of transgenic cells comprising a polynucleotide encoding a chimeric polypeptide, the polynucleotide comprising at least a first nucleic acid sequence encoding a polypeptide of interest of the chimeric polypeptide and at least a second nucleic acid sequence encoding an essential protein of the chimeric polypeptide, and wherein at least 10% of the plurality of transgenic cells are genetically optimized such that the expression of the polypeptide of interest is substantially maintained after a period of at least 80 generations.

According to another aspect, there is provided a method for producing a polynucleotide molecule encoding a chimeric polypeptide comprising a polypeptide of interest, the method comprising: (a) generating or receiving a nucleic acid sequence comprising a coding region encoding a chimeric polypeptide, wherein the coding region comprises a 5′ region encoding the polypeptide of interest and a 3′ region encoding an essential gene of a target cell, optionally wherein the coding region comprises a region between the 5′ region and the 3′ region encoding a linker; (b) expressing the nucleic acid sequence in the target cell under conditions sufficient for expression of the chimeric polypeptide, wherein the target cell is devoid of an endogenous functional form of the essential protein; (c) culturing the target cell expressing the nucleic acid sequence for a time sufficient to determine if the chimeric protein can replace an essential function of the endogenous functional form of the essential protein; and (d) selecting the nucleic acid sequence if the chimeric polypeptide can replace the essential function; thereby producing a polynucleotide molecule encoding a chimeric polypeptide.

According to another aspect, there is provided a method for genetically optimizing the expression of a polypeptide of interest such that it is substantially maintained in least 10% of a plurality of transgenic cells after a period of at least 80 generations, the method comprising: (a) receiving a nucleic acid sequence comprising a coding region encoding a polypeptide of interest; (b) generating a coding sequence encoding a chimeric polypeptide comprising the polypeptide of interest and an essential gene of a target cell optimized thereto for the generation of a chimeric polypeptide in the target cell, wherein the optimized comprises: a modified GC content, at least one less mutation hotspot, an modified codon usage for optimized expression of the coding sequence in the target cell, at least one less epigenetic hotspot, or any combination thereof, compared to a wildtype nucleic acid sequence encoding any one of: the polypeptide of interest, the essential gene of the target cell, and both; and wherein the chimeric polypeptide retains an essential function of the essential gene in the target cell; and (c) expressing the chimeric polypeptide in the plurality of transgenic cells.

According to another aspect, there is provided a computer program product, comprising a non-transitory computer-readable storage medium having program code embodied thereon, the program code executable by at least one hardware processor to: (a) receiving a nucleic acid sequence comprising a coding region encoding a polypeptide and selecting a nucleic acid sequence encoding an essential gene of a target cell optimized thereto for the generation of a chimeric polypeptide; and (b) generating a coding sequence encoding the chimeric polypeptide being genetically optimized such that is comprises: a modified GC content, at least one less mutation hotspot, an optimized codon usage according to the preference of the target cell, at least one less epigenetic hotspot, or any combination thereof, compared to a wildtype nucleic acid sequence encoding any one of: the polypeptide, the essential gene of the target cell, and both, and wherein the chimeric polypeptide retains an essential function of the essential gene in the target cell.

In some embodiments, the at least first nucleic acid sequence encoding the polypeptide of interest is genetically optimized such that is comprises: a modified GC content, at least one less mutation hotspot, an optimized codon usage according to the preference of the transgenic cells, at least one less epigenetic hotspot, or any combination thereof, compared to a wildtype nucleic acid sequence encoding the polypeptide of interest.

In some embodiments, the at least one mutation hotspot comprises a simple sequence repeat (SSR), a repeated mediated deletion (RMD), or both.

In some embodiments, the epigenetic hotspot comprises a methylation site.

In some embodiments, substantially maintained comprises an expression of the polypeptide of interest after a period of at least 80 generations being at least 75% of the expression level of the polypeptide of interest after 1 generation.

In some embodiments, at least 20% of the plurality of transgenic cells are genetically optimized such that the expression of the polypeptide of interest after a period of at least 80 generations is at least 75% of the expression level of the polypeptide of interest after 1 generation.

In some embodiments, the transgenic cells are solitary cells.

In some embodiments, the chimeric polypeptide comprises the polypeptide of interest N-terminally to the essential protein of the transgenic cells.

In some embodiments, the polypeptide of interest and the essential protein are not the same protein.

In some embodiments, the essential protein is essential for: cell vitality, cell mitosis, cell metabolism, cell differentiation, DNA polymerization, RNA transcription, protein translation, housekeeping activity, and any combination thereof, of any one of the plurality of transgenic cells or replication, packaging, host cell recognition, infection efficiency, or any combination thereof, of a virus so as to infect the plurality of transgenic cells.

In some embodiments, the essential protein is the complete protein or a fragment thereof, comprising an essential function.

In some embodiments, removal of expression of the essential protein from the transgenic cells induces death of the transgenic cells, replication arrest of the transgenic cells, or both.

In some embodiments, the chimeric polypeptide further comprises a linker sequence between the first amino acid sequence and the second amino acid sequence.

In some embodiments, the linker sequence comprises 2 to 50 amino acids.

In some embodiments, the linker is a flexible linker.

In some embodiments, the linker is concatenated to the first amino acid sequence of the chimeric polypeptide and to the second amino acid sequence of the chimeric polypeptide, thereby providing optimal folding of both the first amino acid sequence of the chimeric polypeptide and the second amino acid sequence of the chimeric polypeptide.

In some embodiments, provides optimal folding comprises: reduces folding disturbance, increases spatial separation, restores folding, or any combination thereof, of the first amino acid sequence and the second amino acid sequence of the chimeric polypeptide.

In some embodiments, the linker is a 2A peptide.

In some embodiments, the 2A peptide is selected from: P2A, T2A, E2A and F2A.

2A peptides, including specific sequence, would be apparent to one of ordinary skill in the art, and are disclosed in Kim et al., 2011 (PLoS).

In some embodiments, the chimeric polypeptide further comprises a protein-localization sequence.

In some embodiments, the protein-localization sequence is operably linked to the polypeptide of interest, and optionally wherein the protein-localization sequence is upstream of the first sequence.

In some embodiments, the localization is to a cellular location to which the essential protein localizes.

In some embodiments, the cellular location is selected from the group consisting of: nucleus, nucleolus, endoplasmic reticulum (ER), plasma membrane (PM), peroxisome, lysosome, centromere, centrosome, spindle, multivesicular bodies (MVBs), mitochondria, and exosome.

In some embodiments, the chimeric polypeptide further comprises a tag.

In some embodiments, the chimeric polypeptide further comprises a protease recognition site between the first amino acid sequence and the second amino acid sequence.

In some embodiments, any one of the transgenic cells are devoid of an endogenous functional form of the essential protein, optionally wherein any one of the transgenic cells are devoid of an endogenous essential protein.

In some embodiments, any one of the transgenic cells comprises an endogenous genome being devoid of a gene encoding the functional essential protein, optionally wherein the endogenous genome is devoid of a gene encoding the essential protein.

In some embodiments, the transgenic cells are yeast cells.

In some embodiments, replacing an essential function of the endogenous functional form of the essential protein is replacing all essential functions of the endogenous functional form of the essential protein.

In some embodiments, determining comprises determining if the cell dies, enters replication arrest, or both.

In some embodiments, the method further comprises culturing the target cell expressing the nucleic acid sequence for a time sufficient to determine if the cell can lose expression of the protein of interest and still retain the essential function, and not selecting the polynucleotide molecule if the expression can be lost while retaining the essential function.

In some embodiments, expressing comprises transferring an expression vector comprising the nucleic acid sequence into the target cell; or modifying a genome of the target cell to include the nucleic acid sequence.

In some embodiments, method is a method of producing a polynucleotide molecule encoding a polypeptide of interest with increased genetic stability.

In some embodiments, increased genetic stability is as compared to a polynucleotide molecule encoding a polypeptide of interest unlinked to the essential protein.

In some embodiments, selecting further comprises selecting a sequence encoding a linker being concatenated to the polypeptide and to the essential gene of the target cell, of the chimeric polypeptide.

In some embodiment, the method further comprises a step proceeding step (c), comprising determining the expression level of the polypeptide of interest of the chimeric polypeptide in the plurality of transgenic cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the study of the following detailed description.

### BRIEF DESCRIPTION OF THE FIGURES

FIG. 1 includes a flow chart of a non-limiting outline of a method for constructing genetic circuits contemplated by the present invention.

FIG. 2 includes a schematic representation of a non-limiting example of a proposed solution—interlocking a gene of interest (hence referred to as a “target gene”) upstream to an essential gene in the host's genome, under the same promoter. The essential gene serves as a stabilizing element that prevents most mutations to the target gene and promotes its stability. The combination of target gene, essential gene and linker is termed the “combined construct”.

FIG. 3 includes a vertical bar graph showing the change in fluorescence (% of original fluorescence) after 180 generations, relative to day one. The “GFP alone” bar shows florescence change from a target gene introduced into the host genome without being linked with an essential host gene (control); all other bars represent the level of florescence from target genes which have been linked to various essential host genes (Fol3, Nus1, Dfr1, Sec2, Ram2, Tsc13, Ceg1, Sqt1, Kap95, and Cdc9. All but one of these show significant improvements in florescence compared to the non-linked control.

FIG. 4 includes a graph showing Spearman correlation of −0.19 between initial fluorescence and the maintained fluorescence at the end of the experiment. P value=0.57.

FIG. 5 includes a non-limiting flow chart of describing a co-stability prediction model development, as described herein.

FIG. 6 includes a graph showing the correlation between empirical data and predictions made by the herein disclosed sTAUbility Enhancer software.

FIGS. 7A-7B include graphs showing a non-limiting example of a graphic view of the change in the disorder profile for a construct of a chosen linker, (7A) target gene and (7B) essential gene.

FIG. 8 includes a table presenting a non-limiting example of a motif. The first line details the motif name. The second line details, in order, the alphabet length, the length of the motif (how many nucleotides in the methylation site), number of source sites, and E-value. The columns are ordered as ACGT, the rows by the nucleotide index within the motif. Each row gives a probability distribution for the appropriate index.

FIG. 9 includes a non-limiting scheme showing conversation score analysis scheme. Fifteen (15) Saccharomyces cerevisiae conserved genes were analyzed. For each gene, a per-nucleotide conversation score was calculated using the ConSurf tool. Utilizing the ESO, evolutionary unstable areas were marked (asterisks). The average conversation score was then calculated for the entire protein (4.7, left) and the marked areas (2.4, right). The scores were compared to find statistical significance.

FIG. 10 include an illustration of a non-limiting example of a selection process to the most-fit variant in a population of genetically modified microorganisms, resulting in their evolutionary instability.

FIGS. 11A-11B include illustrations of non-limiting examples of possible molecular hotspots within a given genetic sequence, detected with the herein disclosed evolutionary stability optimizer (ESO). (11A) mutational hotspots, (11B) and epigenetic hotspots. The mutational hotspots include simple sequence repeats (SSR), which are repeating short sequences. Due to polymerase slippage mistakes, a short sequence can be added or deleted. Another type of mutational hotspot is repeat mediated deletions (RMD), where longer sequences appear in different parts of the gene, and a misread would cause a deletion of the intermediate sequence. The epigenetic hotspots considered are methylation sites, where the gene is statistically compared to known methylation sites. The attachment to methyl groups can cause a change in the protein's folding, potentially leading to lesser or no gene expression.

FIG. 12 include a non-limiting example of an illustration of the acceptable input and output by the ESO. The ESO takes an input of the format appears in the top left block and returns an output of a similar structure (right block). Within each output folder, one can find tables (in CSV formats) detailing the sites found, and an optimization report (zip format) including the files in the bottom left block: the final sequence in GeneBank format, the sequenticon of the sequence before and after the changes, and the summary of the changes. The icons representing the sequencing files in the output and input folders are examples of sequenticons: unique identifiers for each sequence.

FIGS. 13A-13B include non-limiting examples of illustrations of the ESO main screen (13A) and optimization screen (13B). Within the main screen, the user selects an input directory, the sequences within which will be analyzed, and the output directory, where the results will be stored. In addition, one may define whether to consider methylation or not, how many sites to consider, and whether to calculate an optimized sequence or just return a list of sites to be corrected. Within the optimization screen, the user may define which organism and which method will be used to optimize codon usage, the bounds on GC content, and the ORF indices of the different sequences. If more than one sequence appears within a file, they will be given a running index called Seq Num. Note that the ORF length must be divisible by 3 for codon optimization.

FIG. 14 includes a vertical bar graph showing normalized average conservation score of the entire protein compared to the normalized average conservation score of areas predicted by the ESO to be genetically unstable. Conservation score is normalized in each protein according to the most nsereved residue. The average scores refer to 15 conserved proteins. The conservation score differs in significant value of <0.0001, studet t test.

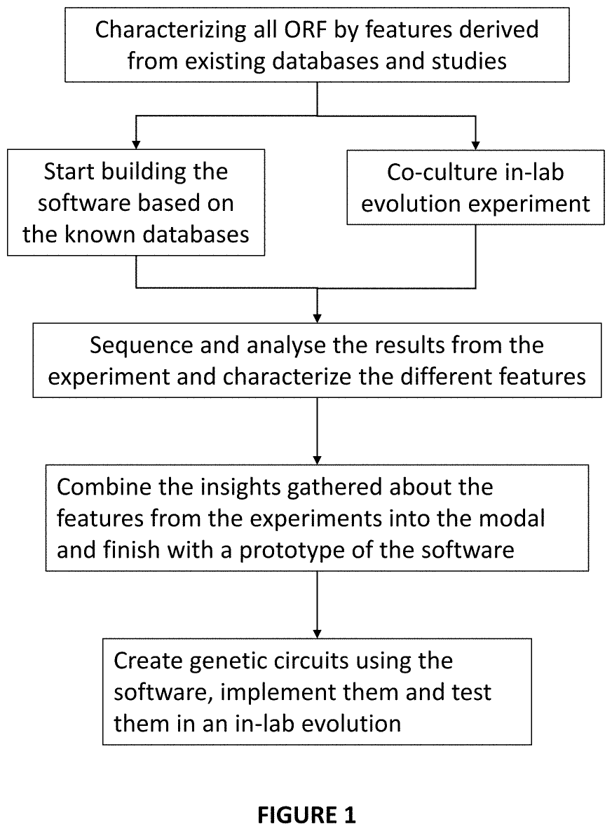

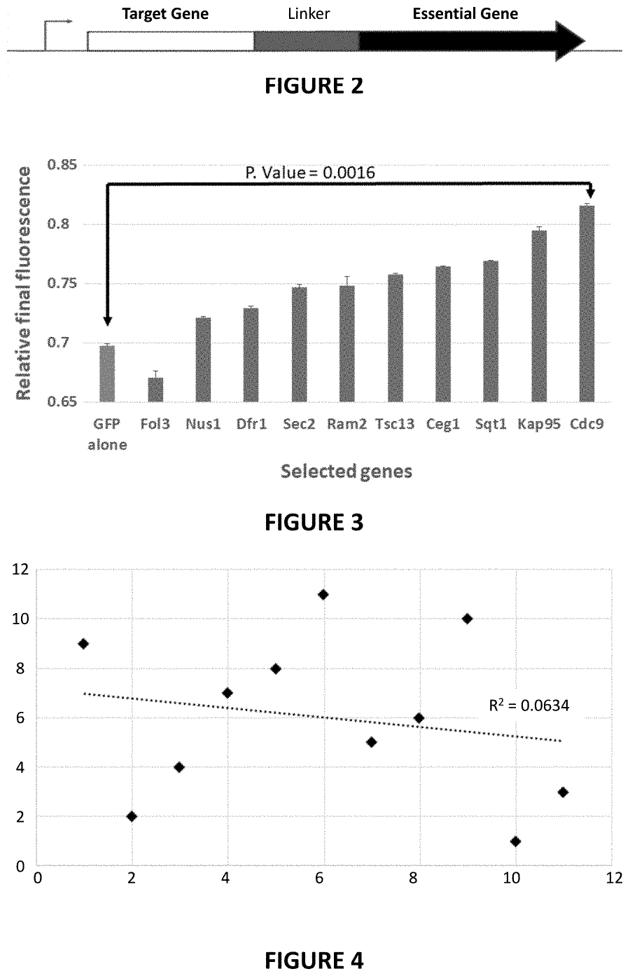

### DETAILED DESCRIPTION

### Chimeric Polypeptide

In some embodiments, there is provided a chimeric polypeptide comprising a first amino acid sequence and a second amino acid sequence, wherein the first amino acid sequence is an amino acid sequence of a polypeptide of interest and the second amino acid sequence is a sequence of an essential protein of a target cell.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the first amino acid sequence is N-terminal to the second amino acid sequence. In some embodiments, the first amino acid sequence is C-terminal to the second amino acid sequence. In some embodiments, the first and second sequences are configured such that reduction in protein production from the first sequence reduces protein production from the second sequence. In some embodiments, a deletion in the first sequence induces a frame shift in the second sequence. In some embodiments, a truncation in the first sequence induces a frame shift in the second sequence. In some embodiments, a deletion in the first sequence induces a deletion of the second sequence (e.g., introducing a premature stop codon, a nonsense mutation, etc.). In some embodiments, a truncation in the first sequence induces a deletion of the second sequence.

In some embodiments, the polypeptide of interest and the essential protein are not the same protein. In some embodiments, the polypeptide of interest and the essential protein are not derived from the same protein. In some embodiments, the polypeptide of interest and the essential protein are not fragments, domains, portions, amino acid stretches, or any combination thereof, of the same protein. In some embodiments, the polypeptide of interest and the essential protein are not encoded by same the gene. In some embodiments, the polypeptide of interest and the essential protein are derived from different species, genus, order, phylum, or kingdom.

The polypeptide of interest may be any protein which a skilled artisan wishes to produce. In some embodiments, the polypeptide of interest is a full protein. In some embodiments, the polypeptide of interests is a fragment of a protein. In some embodiments, the polypeptide of interests is an enzyme. In some embodiments, the polypeptide of interests is an antibody. In some embodiments, the polypeptide of interests is a therapeutic protein. In some embodiments, the polypeptide of interest is a structural protein. In some embodiments, the polypeptide of interest is a scaffold protein. In some embodiments, the polypeptide of interest is a reporter gene. In some embodiments, the polypeptide of interest is a heterologous protein. In some embodiments, the polypeptide of interest is industrially relevant protein. Examples of industrial and pharmaceutically relevant proteins include, but are not limited antibodies, antibody fragments, hormones, interleukins, enzymes, coagulants and vaccines to name but a few. Specific examples of proteins include, but are not limited to, insulin, thyroid hormone, human growth hormone, follicle-stimulating hormone, factor VIII, erythropoietin, granulocyte colony-stimulating factor, alpha-galactosidase A, alpha-L-iduronidase, N-acetylgalactosamine-4-sulfatase, interferon, insulin-like growth factor 1, and lactase. As used herein, the terms “essential gene” or “essential protein” refer to any gene or protein which a cell cannot maintain or properly maintain life in its absence or lack of functionality. In some embodiments, the removal of expression of the essential protein from the target cell induces death of the target cell, replication arrest of the target cell or both.

In some embodiments, a target cell devoid of an essential gene dies. In some embodiments, a target cell comprising an inactive or a dysfunctional essential gene dies. In some embodiments, a target cell devoid of or comprising an inactive or a dysfunctional essential gene has a reduced fitness compared to a native or naïve cell. In some embodiments, fitness comprises cell proliferation, cell differentiation, cell division, DNA replication, RNA transcription, protein translation, energy production, or any combination thereof. In some embodiments, the essential protein is essential for: cell vitality, cell mitosis, cell metabolism, cell differentiation, DNA polymerization, RNA transcription, protein translation, housekeeping activity, or any combination thereof, of the cell as disclosed herein, or a plurality thereof. In some embodiments, the essential protein is essential for replication, packaging, host cell recognition, infection efficiency, or any combination thereof, of a virus suitable for or capable of infecting the cell as disclosed herein, or a plurality thereof.

The terms “essential gene” and “essential protein” are used herein interchangeably.

In some embodiments, the essential protein is the complete protein or a fragment thereof comprising an essential function. In some embodiments, an essential function comprises a functional portion of the protein, a domain, a catalytic domain, a structural domain, a catalytic triad, a binding site, or a recognition site. In some embodiments, an essential function is an enzymatic function. In some embodiments, an essential function is a structural function. In some embodiments, an essential function is a receptor function. In some embodiments, an essential function is a signaling function. In some embodiments, an essential function is a function is cellular replication. Cellular replication is well known in the art, and the various steps and proteins required for replication are well known. The process of prokaryotic replication is described, for example, in “Prokaryotic DNA Replication” by Marians in Annual Review of Biochemistry, 1992, Vol. 61:673-715, herein incorporated by reference in its entirety. The process of eukaryotic replication is described, for example, in “DNA Replication in Eukaryotic Cells” by Bell and Dutta in Annual Review of Biochemistry, 2002, Vol. 71:333-374, herein incorporated by reference in its entirety. In some embodiments, the essential function is cellular metabolism. In some embodiments, cellular metabolism comprises mitochondrial function. The processes involved in metabolic signaling and mitochondrial function is reviewed in “The Multifaceted Contributions of Mitochondria to Cellular Metabolism” by Spinelli and Haigis, 2018, 20:745-754, herein incorporated by reference in its entirety. In some embodiments, the essential function is cell signaling. In some embodiments, the essential function is transcriptional regulation. In some embodiments, the essential protein is a transcription factor. Databases of essential genes by organism are available. For example, a list of essential genes for various organisms can be found at the Database of Essential Genes (DEG) (tubic.tju.edu.cn/deg).

In some embodiments, the chimeric protein retains an essential function of the essential protein. In some embodiments, the chimeric protein retains all essential functions of the essential gene. In some embodiments, the chimeric protein fully retains the essential function. In some embodiments, the chimeric protein retains partial function. In some embodiments, the partial function is sufficient function for a target cell to survive, replicate of both. In some embodiments, the chimeric protein retains an essential interaction of the essential protein. In some embodiments, the chimeric protein retains an essential activity of the essential protein.

In some embodiments, the chimeric polypeptide further comprises a linker sequence. In some embodiments the linker is located between the first amino acid sequence and the second amino acid sequence.

In some embodiments, the linker provides, increases, enables, enhances, any equivalent thereof, or any combination thereof, optimal folding of the first amino acid sequence and the second amino acid sequence of the chimeric polypeptide.

In some embodiments, the linker as disclosed herein reduces folding disturbance, increases spatial separation, restores folding, or any combination thereof, of the first amino acid sequence and the second amino acid sequence of the chimeric polypeptide.

In some embodiments, the linker is chosen based on its suitability to increase folding accuracy, proficiency, efficiency, thermodynamic stability of both the first amino acid sequence and the second amino acid sequence of the chimeric polypeptide.

As used herein, the term “linker” refers to a molecule or macromolecule serving to connect the different moieties of the chimeric polypeptide of the invention, e.g., the polypeptide of interest and the essential protein. In one embodiment, the linker may also facilitate other functions, including, but not limited to, preserving biological activity, maintaining sub-units and/or domains interactions, and others. In some embodiments, the linker is a flexible linker or a rigid linker.

In some embodiments, the amino acid sequence of the linker and/or the amino acid sequence of any one of the polypeptide of interest, the essential protein, and the chimeric polypeptide of the invention, are co-modified so to improve: protein folding, expression, function, or any combination thereof. In some embodiments, amino acid sequence co-modification comprises preventing the common or native folding of the polypeptide of interest, the essential protein, or both. In some any amino acid sequence modification is applicable as long as the provided chimeric polypeptide is functional. In some embodiments, the linker is a flexible linker. In some embodiments, the linker is a rigid linker.

In some embodiments, the linker comprises at least 2, 5, 7, 9, 10, 15, 20, 25, 30, 35, 40, 45, 50 amino acids, or any value and range therebetween. Each possibility represents a separate embodiment of the invention. According to some embodiments, the linker comprises 2-20, 5-50, 1-15, 3-28, 4-44, or 2-50 amino acids. Each possibility represents a separate embodiment of the invention. In some embodiments, the linker is of a sufficient length such that the protein of interest does not impair the functionality of the essential protein. In some embodiments, the functionality is an essential function. In some embodiments, the linker is configured to prohibit interference of the protein of interest in a functionality of the essential protein. In some embodiments, the linker is configured to allow separate folding of the protein of interests and the essential protein. A skilled artisan will appreciate that in order for the essential protein to function it must fold as normal and that the amino acid sequence of the protein of interest could interact with the essential protein and cause misfolding. However, as folding commences immediately upon exit from the ribosome, having a space (the linker) between the two proteins reduces the chances of misfolding.

In some embodiments, the chimeric polypeptide further comprises a protein-localization sequence. In some embodiments, a protein localization sequence is or comprises an amino acid sequence which translocates a protein comprising the protein localization sequence to a cellular location. In some embodiments, a protein comprising the protein localization sequence is more likely to be present, function, located, or identified in a particular or specific cellular compartment over other cellular components, compartments, or areas of the cell.

In some embodiments, the protein-localization sequence is operably linked to the polypeptide of interest. In some embodiments, the protein-localization sequence is located upstream of the polypeptide of interest sequence. In some embodiments, the protein-localization sequence is operably linked to the polypeptide of interest, optionally wherein the protein-localization sequence is upstream of the polypeptide of interest sequence.

In some embodiments, the chimeric protein localizes to the cellular location to which the essential protein localizes. In some embodiments, the localization is to a cellular location to which the essential protein is or more likely to be is present, functions, located, or can be identified particularly or predominantly. In one embodiment, the localization is to a cellular location to which the essential protein localizes.

In some embodiments, the cellular location is selected from: nucleus, nucleolus, endoplasmic reticulum (ER), plasma membrane (PM), peroxisome, lysosome, centromere, spindle, centrosome, multivesicular bodies (MVBs), mitochondria, Golgi apparatus, and exosome.

In some embodiments, the chimeric polypeptide further comprises a tag.

The tag may be any tagging molecule or moiety known in the art, including, but not limited to a fluorescent tag, a short peptide tag or a protein tag. Non-limiting examples of fluorescent tags include GFP tags, CFP tags, YFP tags, RFP tags, CY3 tags, CY5 tags, CY7 tags, fluorescein tags, and ethidium bromide tags. Non-limiting examples of peptide tags include Myc tag, His tags, FLAG tags, HA-tags, SBP tags, Non-limiting examples of protein tags include glutathione tags, BCCP tags, MBP tags, and protein A tags. In some embodiments, the tag is cleavable. In some embodiments, the tag is used during production of the molecule and removed or cleaved before administration to a subject. In some embodiments, the tag is used for protein purification. In some embodiments, the molecule comprises tandem tags. In some embodiments, the tandem tags are used for tandem affinity purification. In some embodiments, the molecule comprises more than one copy of a given tag. It is well known in the art that some tags can be used as repeated tags, such as 3×FLAG and 6×His.

In some embodiments, the chimeric polypeptide further comprises a protease recognition site. In some embodiments, the protease recognition site is located between the polypeptide of interest and the essential protein. In some embodiments, the linker comprises a protease recognition site. In some embodiments, the protease recognition site is a protease site. In some embodiments, the protease recognition site is a proteolytic cleavage site.

As used herein, the term “protease” refers to a group of proteins capable of activating other inactivated target proteins by modifying the inactivated proteins, e.g., by means of proteolytic cleavage at a specific site or motif. In some embodiments, the protease digests one polypeptide sequence into at least 2 distinct polypeptide sequences. In some embodiments, the protease digests the chimeric polypeptide of the invention, thereby cleaving the polypeptide of interest off the essential protein.

The terms “specific site”, “motif”, “cleavage site”, “proteolytic site” and “protease recognition site” are used herein interchangeably.

### Polynucleotide Molecule

According to some embodiments, there is provided a polynucleotide molecule comprising a sequence encoding the chimeric polypeptide of the invention.

The terms “polynucleotide”, “polynucleotide sequence”, “nucleic acid sequence”, and “nucleic acid molecule” are used interchangeably herein. These terms encompass nucleotide sequences and the like. A polynucleotide may be a polymer of RNA, DNA, or a hybrid thereof, that is single- or double-stranded, that optionally contains synthetic, non-natural or altered nucleotide bases.

In some embodiments, the polynucleotide comprises increased genetic stability. In some embodiments, the increased genetic stability is when the polynucleotide is expressed in a target cell. In some embodiments, the increased genetic stability is when the polynucleotide is expressed in a cell devoid of a functional form of the essential protein. In some embodiments, the functional form is an endogenous functional form. In some embodiments, the increased genetic stability is when the polynucleotide is expressed in a cell devoid of the essential protein. In some embodiments, increased is as compared to a polynucleotide molecule encoding the polypeptide of interest unlinked to the essential protein. In some embodiments, increased is as compared to a polynucleotide molecule encoding the polypeptide of interest alone. In some embodiments, increased is as compared to a polynucleotide molecule encoding the polypeptide of interest not as part of a chimeric polypeptide. In some embodiments, increased is as compared to a polynucleotide molecule encoding the polypeptide of interest not as part of a chimeric polypeptide of the invention. In some embodiments, increased genetic stability comprises a decreased mutational rate. In some embodiments, the decreased mutational rate is the mutational rate of the polypeptide of interest. In some embodiments, the decreased mutational rate is the mutational rate of a regulatory element of the nucleic acid molecule. In some embodiments, the decreased mutational rate is the mutational rate of a regulatory element. In some embodiments, the decreased mutational rate is the mutational rate of an element that regulates expression of the chimeric polypeptide and/or the polypeptide of interest. In some embodiments, the regulatory element is the promoter. In some embodiments, the decreased is as compared to a nucleic acid molecule comprising a regulatory element that regulates expression of a polypeptide of interest that is not linked to an essential gene, not part of a chimeric polypeptide, or not part of a chimeric polypeptide of the invention. It will be understood by a skilled artisan that by putting the essential gene after the polypeptide of interest and/or under the control of the same promoter any mutation that is generated that also impacts the essential gene will not be passed on to future cells or if passed on will confer inferior survival and eventually be overwhelmed by superior cells. This process decreases the incidence of mutation in the whole construct. In some embodiments, decreased mutational rate is decreased incidence of mutations that are passed on. In some embodiments, decreased mutational rate is decreased incidence of mutations that are retained in a population of cells.

In some embodiments, the polynucleotide molecule further comprises at least one regulatory sequence. In some embodiments, the regulatory sequence is operably linked to the sequence encoding the chimeric polypeptide.

As used herein, the term “operably linked” is intended to mean that the nucleotide sequence of interest is linked to the regulatory element or elements in a manner that allows for expression of the nucleotide sequence (e.g., in an in-vitro transcription/translation system or in a host cell when the vector is introduced into the host cell).

In some embodiments, the regulatory sequence is a promoter sequence.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, the promoter sequence comprises an endogenous promoter sequence of the gene encoding the essential protein. As used herein, the term “endogenous” and “endogenously” refers to that the essential gene is under the regulation of the promoter in a target cell devoid of the chimeric polypeptide of the invention and/or a polynucleotide encoding the chimeric polypeptide. In some embodiments, the endogenous promoter and the essential gene (e.g., encoding the essential protein) are parts of the same gene and/or are located in the same genomic DNA region.

In some embodiments, the promoter sequence is a heterologous promoter sequence. As used herein, the terms “heterologous” or “heterologous expression” refers to that polypeptide of interest originates from a different cell type or a different species from the target cell (e.g., configured to expression).

In some embodiments, the chimeric protein is encoded by a single reading frame. In some embodiments, a single promoter regulates expression of the single reading frame. It will be understood by a skilled artisan that by having a single promoter a promoter mutation that would abolish or reduce transcription of the target protein would also inherently abolish or reduce transcription of the essential protein. Thus, a mutation that might improve cellular fitness by reducing the load of producing the exogenous target protein would also reduce cellular fitness by reducing the essential protein.

In some embodiments, there is provided an expression vector comprising the herein disclosed polynucleotide molecule. In some embodiments, the polynucleotide molecule is an expression vector. In some embodiments, the expression vector is configured to express in a target cell. In some embodiments, the target cell lacks the essential protein. In some embodiments, the polynucleotide molecule is an expression vector configured to express in a target cell, wherein optionally, the target cell lacks the essential protein. In some embodiments, the vector is a DNA vector. In some embodiments, the vector is an RNA vector. In some embodiments, the vector further comprises any elements required for expression of the chimeric protein in a target cell.

In some embodiments, the expression vector is a prokaryotic expression vector. In some embodiments, the prokaryotic expression vector comprises any sequences necessary for expression of the protein encoded by the nucleic acid molecule of the invention in a prokaryotic cell. In some embodiments, the expression vector is a eukaryotic expression vector.

In some embodiments, the expression vector is a mammalian expression vector. Mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, the expression vector contains regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, a recombinant viral vector, which offers advantages such as lateral infection and targeting specificity, is used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

General methods in molecular and cellular biochemistry, such as methods useful for carrying out DNA and protein recombination, as well as other techniques described herein, can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

In one embodiment, the expression vector is a plant expression vector. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the chimeric polypeptide), the expression construct of the present invention can also include sequences engineered to optimize stability, production, purification, yield or activity of the expressed polypeptide.

The term “expression” as used herein refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, transformation, viral infection, or direct alteration of the cell's genome. In some embodiments, the gene is in an expression vector such as plasmid or viral vector.

Recombinant expression vectors generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence that allows for expression of the nucleotide sequence (e.g. in an in vitro transcription/translation system or in a host cell when the vector is introduced into the host cell).

As used herein the term “in vitro” refers to any process that occurs outside a living organism. As used herein the term “in-vivo” refers to any process that occurs inside a living organism. In one embodiment, “in-vivo” as used herein is a cell within an intact tissue or an intact organ.

In some embodiments, the polynucleotide comprises a nucleic acid which is modified so as to improve translation efficacy. In some embodiments, modification so as to provide improved translation efficacy comprises codon optimization.

The term “codon optimization” refers to a process directed to improving heterologous gene expression and increase the translational efficiency of a recombinant gene of interest by modifying the nucleic acid sequence of the recombinant gene of interest so as to accommodate codon bias according to the host cell or organism.

In some embodiments, codon optimization does not alter the amino acid sequence of the polypeptide of interest. In some embodiments the poly nucleotide is modified by means of mutagenesis. In some embodiments, the polynucleotide comprises at least one mutation compared to the wildtype sequence from which it is derived. In some embodiments, the mutation is a silent mutation. In some embodiments, the mutation is a missense mutation. In some embodiments, the mutation is not a nonsense mutation. In some embodiments, the mutation is any mutation which improves protein production rates, yields, stability, or any combination thereof, wherein the produced protein is a functional chimeric polypeptide of the invention.

In some embodiments, the polynucleotide sequence is modified so as to include silent mutations. In some embodiments, the polynucleotide sequence encoding the polypeptide of interest, the essential protein, or both (e.g., the chimeric polypeptide of the invention) comprises a silent mutation. In some embodiments, the regulatory sequence as disclosed herein, comprises a silent mutation. In some embodiments, a silent mutation increases or enhances (e.g., optimizes) expression, improves protein folding and stability, or both, of the polypeptide of interest, the essential protein, or both. In some embodiments, a silent mutation as disclosed herein, comprises any mutation which enables the production of a functional chimeric polypeptide of the invention which increases the speed of the ribosome during co-translational folding.

In some embodiments, the mutation improves the stability of the polynucleotide sequence of the invention, e.g., encodes the chimeric polypeptide of the invention. In some embodiments, the introduced mutation reduces the rate of subsequent mutagenesis in the polynucleotide of the invention. In some embodiments, the introduced mutation prevents subsequent mutagenesis in the polynucleotide of the invention. In some embodiments, the introduced mutation reduces or eliminates the mutagenesis probability or potential of mutagenesis prone sequences. In some embodiments, the introduced mutation modifies mutagenesis prone sequences into mutagenesis non-prone or indifferent sequences. In some embodiments, the mutation is introduced into sequences which further promote subsequent mutations which hamper, reduce, inhibit, prevent, eliminate, or any combination thereof, the transcription, translation, or both, of the chimeric polypeptide of the invention.

A person with skill in the art will appreciate that a gene can also be expressed from a nucleic acid construct administered to the individual employing any suitable mode of administration described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex vivo gene therapy).

### Cells

According to some embodiments, there is provided a composition comprising a plurality of cells comprising a polynucleotide encoding a chimeric polypeptide, the polynucleotide comprising at least a first nucleic acid sequence encoding a polypeptide of interest of the chimeric polypeptide and at least a second nucleic acid sequence encoding an essential protein of the chimeric polypeptide.

In some embodiments, at least 5%, at least 10%, at least 20%, at least 30%, at least 40%, at least 50%, at least 60%, at least 70%, 80%, at least 90%, at least 95%, at least 99%, 100% of the plurality of transgenic cells, or any value and range therebetween, are genetically optimized such that the expression of the polypeptide of interest is substantially maintained after a period of at least 50 generations, at least 60 generations, at least 70 generations, at least 80 generations, at least 90 generations, at least 100 generations, at least 150 generations, at least 170 generations, at least 200 generations, or at least 500 generation, or any value and range therebetween. Each possibility represents a separate embodiment of the invention.

As used herein, the term “generation” refers to a “cell generation” or a “cell cycle” and is meant to be understood as an integer related to the number of cell cycles or duplications of cells in a composition undergoing logarithmic/exponential growth, as would be apparent to one of ordinary skill in the art of cell biology.

As used herein, the terms “generations”, “cell cycles”, and “cell duplications” are interchangeable.

In some embodiments, “substantially maintained” is compared to the expression level of a polypeptide of interest being unfused or unlinked to an essential protein. In some embodiments, “substantially maintained” is compared to the expression level of a polypeptide of interest not in a chimeric polypeptide. In some embodiments, “substantially maintained” is compared to the expression level of a polypeptide of interest in a chimeric polypeptide devoid of an essential protein.

In some embodiments, “substantially maintained” comprises ±1%, ±2%, ±4%, ±5%, ±7%, ±8%, ±10%, ±12%, ±15%, ±20%, ±25%, ±35%, ±40%, ±50%, ±55%, or ±60%, compared to a control polypeptide of interest. Each possibility represents a separate embodiment of the invention.

In some embodiments, a control polypeptide of interest comprises polypeptide of interest being unfused or unlinked to an essential protein. In some embodiments, a control polypeptide of interest comprises a polypeptide of interest not in a chimeric polypeptide. In some embodiments, a control polypeptide of interest comprises a polypeptide of interest in a chimeric polypeptide devoid of an essential protein.

In some embodiments, 10-20%, 5-30%, 9-35%, 15-40%, 30-80%, or 10-100% of the plurality of transgenic cells, are genetically optimized such that the expression of the polypeptide of interest is substantially maintained after a period of 50-100 generations, 70-150 generations, 65-170 generations, 80-290 generations, or 90-500 generation. Each possibility represents a separate embodiment of the invention.

In some embodiments, the at least first nucleic acid sequence encoding the polypeptide of interest is genetically optimized.

In some embodiments, genetic optimization comprises one or more molecular modifications of the at least first nucleic acid sequence. In some embodiments, the one or more molecular modifications improve, increase, enhance, or any combination thereof, the expression level of the polypeptide of interest. In some embodiments, the one or more molecular modifications, improve, increase, enhance, or any combination thereof, the genetic and/or genomic stability of a cell expressing the polypeptide of interest. In some embodiments, the type and/or location of the one or more molecular modifications is determined according to herein disclosed method. In some embodiments, determination of the type and/or location of the one or more molecular modifications is enabled by the computer program product disclosed herein.

In some embodiments, the one or more molecular modification is introduced into a wildtype sequence or a background reference sequence encoding the polypeptide of interest. In some embodiments, the one or more molecular modification is artificially introduced into a wildtype sequence or a background reference sequence encoding the polypeptide of interest. In some embodiments, the one or more molecular modification is introduced into a wildtype sequence or a background reference sequence encoding the polypeptide of interest in vitro. Methods of molecular biology achieving sequence modifications, e.g., introduction of a point mutation, deletion of desired sequence, etc., are common and would be apparent to one of ordinary skill in the art, non-limiting examples of such methods, include, but are not limited to PCR (e.g., using primers introducing or removing a mutation from the amplicon), etc.

In some embodiments, the one or more molecular modification comprises: a modified GC content, at least one less mutation hotspot, an optimized codon usage according to the preference of the transgenic cells, at least one less epigenetic hotspot, or any combination thereof. In some embodiments, the method comprises: modifying the GC content, removing, deleting, altering the sequence comprising, or any combination thereof, the at least one mutation hotspot, optimizing codon usage according to the preference of the transgenic cells, removing, deleting, altering the sequence comprising, or any combination thereof, the at least one epigenetic hotspot, or any combination thereof.

In some embodiments, the one or more molecular modification is compared to a wildtype sequence or a background reference sequence.

In some embodiments, “modify” or “modifying” comprises increasing or enhancing.

In some embodiments, modify” or “modifying” comprises reducing or lowering.

In some embodiments, the method comprises increasing the GC content the at least first nucleic acid sequence encoding the polypeptide of interest.

In some embodiments, increasing or enhancing is at least 5%, at least 15%, at least 35%, at least 50%, at least 75%, at least 100%, at least 200%, at least 350%, at least 500%, at least 750%, or at least 1,000% increase, or any value and range therebetween. Each possibility represents a separate embodiment of the invention. In some embodiments, increasing or enhancing is 5-100%, 15-200%, 30-475%, 50-500%, 75-650%, 100-900%, 200-750%, 350-800%, 500-1,250%, or 750-1,500% increase. Each possibility represents a separate embodiment of the invention.

In some embodiments, decreasing or lowering is at least 5%, at least 15%, at least 35%, at least 50%, at least 75%, or at least 100% decrease, or any value and range therebetween. Each possibility represents a separate embodiment of the invention. In some embodiments, decreasing or lowering is 5-10%, 15-50%, 30-75%, 25-85%, 75-100%, 10-90%, 20-75%, 35-80%, or 50-100% decrease. Each possibility represents a separate embodiment of the invention.

In some embodiments, the at least one mutation hotspot comprises a simple sequence repeat (SSR), a repeated mediated deletion (RMD), or both.

In some embodiments, the epigenetic hotspot comprises a methylation site.

In some embodiments, substantially maintained comprises an expression of the polypeptide of interest after a period of at least 20 generations, at least 60 generations, at least 100 generations, at least 150 generations, at least 190 generations, at least 200 generations, at least 250 generations, at least 500 generations, or at least 1,000 generations, being at least 50%, at least 60%, at least 75%, at least 85%, at least 90%, at least 95%, or at least 99% of the expression level of the polypeptide of interest after 1 generation, or any value and range therebetween. Each possibility represents a separate embodiment of the invention.

In some embodiments, substantially maintained comprises an expression of the polypeptide of interest after a period of 20-150 generations, 30-130 generations, 40-250 generations, 150-450 generations, 190-500 generations, 200-700 generations, 250-1,000 generations, being 50-100%, 60-99%, 75-97%, or 85-100% of the expression level of the polypeptide of interest after 1 generation. Each possibility represents a separate embodiment of the invention.

In some embodiments, the cells are transgenic cells. In some embodiments, the cells comprise at least one transgene. In some embodiments, the cells are solitary cells.

As used herein, the term “solitary cells” encompasses any form of cells that are not actively and/or constitutively adhere to one another. In some embodiments, solitary cells are in a suspension. In some embodiments, solitary cells are in a layer form (e.g., seeded on a surface, such as a plate).

According to some embodiments, the composition disclosed herein comprises a plurality of transgenic solitary cells. In some embodiments, the composition is devoid of tissue fragments or organs. In some embodiments, the composition is devoid of cell aggregates comprising at least 10 cells, at least 50 cells, at least 100 cells, at least 1,000 cells, or any value and range therebetween. Each possibility represents a separate embodiment of the invention.

According to some embodiments, there is provided a cell comprising: (a) the chimeric polypeptide of the invention; or (b) the herein disclosed polynucleotide molecule. In some embodiments, the cell is the target cell.

The term “target cell” encompasses any cell configured to expressing the chimeric polypeptide of the invention. In some embodiments, the cell is a bacterial cell. In some embodiments, the cell is a fungal cell. In some embodiments, the cell is a yeast cell. In some embodiments, the cell is a mammalian cell.

In some embodiments, the cell is devoid of an endogenous functional form of the essential protein. In some embodiments, the cell is devoid of the endogenous essential gene, mRNA transcribed therefrom, protein translated therefrom, or any combination thereof.

In some embodiments, the cell is devoid of a gene encoding the functional essential protein.

In some embodiments, the genome of the cell comprises the sequence of the herein disclosed polynucleotide molecule.

In some embodiments, the polynucleotide sequence is integrated into the genome of the cell.

In some embodiments, the endogenous coding sequence encoding the essential protein is replaced with a sequence encoding the chimeric polypeptide of the invention.

In some embodiments, the genome of the cell is devoid of the endogenous gene encoding the essential protein or comprises a dysfunctional or an inactive form thereof.

Cells lacking essential genes, or with the essential genes under inducible control (i.e. can be turned off and/or on) are known in the art. For example, the SWAp-Tag yeast library is available (see Weill et al., “Genome-wide SWAp-Tag yeast libraries for proteome exploration.” Nat Methods. 2018; 15(8):617-22, herein incorporated by reference in its entirety). Methods of making these libraries are also known, see for example Yofe et al., “One library to make them all: streamlining the creation of yeast libraries via a SWAp-Tag strategy.” Nat Methods. 2016; 13(4):371-8, herein incorporated by reference in its entirety.

### Methods

According to some embodiments, there is provided a method for producing a protein of interest, the method comprising the steps of: (a) introducing into a target cell the polynucleotide molecule disclosed herein, wherein the target cell lacks expression of a functional form of the essential protein; and (b) culturing the target cell under conditions suitable for expression of the chimeric polypeptide; thereby producing a protein of interest.

In some embodiments, the target cell has a reduced expression of a functional form of the essential protein. In some embodiments, the target cell has an expression of a dysfunctional form of the essential protein. In some embodiments, the target cell is devoid of the essential protein. In some embodiments, the dysfunctional cell comprises reduced expression of the essential protein.

In some embodiments, introducing comprises transferring an expression vector comprising the polynucleotide molecule into the target cell; or modifying the genome of the target cell to include a sequence of the polynucleotide molecule. In some embodiments, the transferring is transfection. In some embodiments, the transferring is lipofection. In some embodiments, the transferring is nucleofection. In some embodiments, the transferring is viral infection.

In some embodiments, modifying the genome comprises inserting the chimeric polynucleotide to the genome of the cell. In some embodiments, modifying the genome of the cell comprises excising the endogenous gene encoding the essential protein from the genome of the cell and inserting the polynucleotide into the genome of the cell. In some embodiments, inserting and excising take place in the same genomic site or in different genomic sites in the genome of the target cell. In some embodiments, inserting, excising, or both, is by using at least one programmable engineered nuclease (PEN).

In some embodiments, PEN used according to the method of the invention is any one of a clustered regularly interspaced short palindromic repeat (CRISPR) Class 2 or Class 1 system.

The clustered regularly interspaced short palindromic repeats (CRISPR) Type II system is a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein.

CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and Class 2 may be divided into types II, V, and VI. In some embodiments, CRISPR is CRISPR/Cas9. Any combination with a Cas or modified Cas may be used. Further, methods of designing guide RNAs for CRISPR genome editing are well known in the art and any such method may be employed.

As used herein, “CRISPR arrays” also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. In some embodiments the CRISPR spacers used in the sequence encoding at least one gRNA of the methods and kits of the invention comprise between 10 to 75 nucleotides (nt) each. In some embodiments, the gRNA comprises at least: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or any vale and range therebetween. Each possibility represents a separate embodiment of the invention. In some embodiments, the gRNA comprises 70 to 150 nt. In some specific embodiments the spacers comprise 20 to 35 nucleotides.

In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

In some embodiments, the PEN used by the methods of the invention is a CRISPR Class 2 system. In some embodiments, class 2 system comprises or is a CRISPR type II system.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas 1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of Cas9, Cas1, Cas2 csn2, and Cas4 genes. It should be appreciated that any type II CRISPR-Cas systems may be applicable in the present invention, specifically, any one of type II-A or B.

In some embodiments, the at least one Cas gene used in the method of the invention may be at least one Cas gene of type II CRISPR system (either type II-A or type II-B). In some embodiments, at least one Cas gene of type II CRISPR system used by the method the invention is the Cas9 gene. It should be appreciated that such system may further comprise at least one of Cas1, Cas2, csn2 and Cas4 genes.

In some embodiments, a Cas protein consists or comprise a Cas9 protein.

Double-stranded DNA (dsDNA) cleavage by Cas9 is a hallmark of “type II CRISPR-Gas” immune systems. The CRISPR-associated protein Cas9 is an RNA-guided DNA endonuclease that uses RNA:DNA complementarity to identify target sites for sequence-specific double stranded DNA (dsDNA) cleavage, creating the double strand brakes (DSBs) required for the HDR that results in the integration of the reporter gene into the specific target sequence, for example, a specific region within the genome of the target cell comprising a polynucleotide encoding the endogenous essential protein. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as protospacers. These protospacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity.

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the gRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens.

In some embodiments, the cell expresses the essential protein under a first set of conditions. In some embodiments, the cell does not express the essential protein under a second set of conditions. In some embodiments, culturing according to the method of the invention comprises culturing under the second set of conditions. In some embodiments, the essential protein is inducible, and the inducing element is removed in the second set of conditions. Inducible promoters and agents are well known in the art and any such regulatory control may be used. In some embodiments, the second set of conditions comprises administering an agent that degrades the essential protein.

In some embodiments, the method is a method for indefinitely producing the protein of interest and the method further comprises culturing the cell indefinitely without losing expression of the protein of interest. In some embodiments, the method is for indefinitely producing a functional protein of interest and the method further comprises culturing the cell indefinitely without losing functionality of the protein of interest. In some embodiments, indefinitely is at least 3 days, 5 days, 7 days, 2 weeks, 3 weeks, 4 weeks, 5 weeks, 1 month, 2 months, 3 months, 4 months, 5 months 6 months, 7 months, 8 months, 9 months, 10 months, 11 months, 1 year, 2 years, 3 years, 4 years or 5 years. Each possibility represents a separate embodiment of the invention.

In some embodiments, the method further comprises isolating the produced protein of interest. In some embodiments, the method further comprises extracting the produced protein of interest. In some embodiments, the method further comprises purifying the produced protein of interest. In some embodiments, any one of isolating, extracting, and purifying is from the target cell. In some embodiments, any one of isolating, extracting, and purifying is from the culture medium wherein the target cell is cultured. In some embodiments, any one of isolating, extracting, and purifying is from the target cell and from the culture medium wherein the target cell is cultured.

In some embodiments, isolating comprises cleaving at a protease site. In some embodiments, the protease cleavage site is located between the polypeptide of interest and the essential protein, thereby isolating the polypeptide of interest without the essential protein. In some embodiments, the cleaving comprises contacting the chimeric polypeptide with a protease. In some embodiments, isolating comprises isolating the protein of interest from the essential protein. This separation from the essential protein may be via proteolytic cleavage, for example.

In some embodiments, the target cell is cultured under effective conditions, which allow for the expression of high amounts of the polypeptide of interest, the chimeric polypeptide, or both. In some embodiments, effective culture conditions include, but are not limited to, effective media, bioreactor, temperature, pH and oxygen conditions that permit protein production. In one embodiment, an effective medium refers to any medium in which a cell is cultured to produce the polypeptide of interest, the chimeric polypeptide, or both. In some embodiments, a medium typically includes an aqueous solution having assimilable carbon, nitrogen and phosphate sources, and appropriate salts, minerals, metals and other nutrients, such as vitamins. In some embodiments, the cell can be cultured in conventional fermentation bioreactors, shake flasks, test tubes, microtiter dishes and petri plates. In some embodiments, culturing is carried out at a temperature, pH and oxygen content appropriate for a recombinant or a transformed cell. In some embodiments, culturing conditions are within the expertise of one of ordinary skill in the art.

According to some embodiments, there is provided a method for producing a polynucleotide molecule encoding a polypeptide of interest, the method comprising: (a) generating or receiving a nucleic acid sequence comprising a coding region encoding a chimeric polypeptide, wherein the coding region comprises a 5′ region encoding the polypeptide of interest and a 3′ region encoding an essential gene of a target cell; (b) expressing the nucleic acid sequence in the target cell under conditions sufficient for expression of the chimeric polypeptide, wherein the target cell is devoid of an endogenous functional form of the essential protein; (c) culturing the target cell expressing the nucleic acid sequence for a time sufficient to determine if the chimeric protein can replace an essential function of the endogenous functional form of the essential protein; and (d) selecting the nucleic acid sequence if the chimeric polypeptide can replace the essential function; thereby producing a polynucleotide molecule encoding a chimeric polypeptide.

In some embodiments, the method is a method for producing a chimeric polypeptide comprising the polypeptide of interest. In some embodiments, the method is a method for producing a polynucleotide molecule with increased genetic stability. In some embodiments, increased genetic stability is as compared to a polynucleotide molecule encoding a polypeptide of interest unlinked to the essential protein. In some embodiments, increased genetic stability is as compared to a polynucleotide molecule encoding a polypeptide of interest not part of a chimeric polypeptide. In some embodiments, increased genetic stability is as compared to a polynucleotide molecule encoding a polypeptide of interest not part of a chimeric polypeptide of the invention. In some embodiments, the increased genetic stability is when the polynucleotide molecule is expressed in a target cell.

In some embodiments, the coding region further comprises a region between the 5′ region and the 3′ region. In some embodiments, the region between the 5′ region and the 3′ region encodes a linker.

In some embodiments, replacing an essential function of the endogenous functional form of the essential protein is replacing all essential functions of the endogenous functional form of the essential protein. In some embodiments, replacing an essential function of the endogenous functional form of the essential protein is partially replacing the essential functions of the endogenous functional form of the essential protein. In some embodiments, replacing an essential function of the endogenous functional form of the essential protein is replacing at least one essential function of the endogenous functional form of the essential protein.

In some embodiments, replacing an essential function of the endogenous functional form of the essential protein comprises replacing any essential function of the endogenous functional form of the essential protein which increases the fitness of the target cell comprising the polynucleotide molecule encoding the chimeric polypeptide of the invention, compared to a target cell devoid of the polynucleotide molecule encoding a chimeric polypeptide of the invention.

In some embodiments, determining comprises determining if the cell dies, enters replicative arrest or both.

A method for determining the suitability of a chimeric polypeptide to replace an essential function can be based on a lab evolution experiment, according to which cells are grown for many generation (e.g. a few weeks or months), after which genes are sequence so as to evaluate their stability, as disclosed in the example section hereinbelow.

In some embodiments, the determining is performed in parallel, thereby analyzing numerous target essential genes, as disclosed in the example section hereinbelow, co-culture lab evolution followed by sequencing of all the strains together, e.g., via amplification of the relevant regions and next generation sequencing (NGS), according to which the most suitable essential gene can be selected. The identification of promising essential genes can be further utilized so as to devise prediction models according to which the selection of a polypeptide of interest and an essential gene to be paired (e.g., components of a chimeric polypeptide) can be optimized.

Other methods for determining cell death are common and would be apparent to one of ordinary skill in the art. Non-limiting examples for such methods include, but are not limited to, apoptosis assays, nuclear specific stains, and subsequent microscopy (e.g., DAPI, acridine orange/ethidium bromide), trypan blue, and others.

In some embodiments, the method further comprises culturing the target cell expressing the nucleic acid sequence for a time sufficient to determine if the cell can lose expression of the protein of interest and still retain the essential function, and not selecting the polynucleotide molecule if the expression can be lost while retaining the essential function.

In some embodiments, expressing comprises transferring an expression vector comprising the polynucleotide molecule into the target cell; or modifying a genome of the target cell to include the nucleic acid sequence. In some embodiments, expressing comprises contacting a cell with an expression vector comprising the polynucleotide molecule. In some embodiments, transferring and/or contacting further comprises formulating the nucleic acid sequence with a transfection agent capable of internalizing or carrying the polynucleotide molecule across the target cell membrane.

In some embodiments, there is provided a polynucleotide molecule produced by the method of the invention.

According to some embodiments, there is provided a method for genetically optimizing the expression of a polypeptide of interest such that it is substantially maintained in least 10% of a plurality of transgenic cells after a period of at least 80 generations of the cells, the method comprising: (a) receiving a nucleic acid sequence comprising a coding region encoding a polypeptide of interest; (b) generating a coding sequence encoding a chimeric polypeptide comprising the polypeptide of interest and an essential gene of a target cell optimized thereto for the generation of a chimeric polypeptide in the target cell, wherein the optimized comprises: a modified GC content, at least one less mutation hotspot, an modified codon usage for optimized expression of the coding sequence in the target cell, at least one less epigenetic hotspot, or any combination thereof, compared to a wildtype nucleic acid sequence encoding any one of: the polypeptide of interest, the essential gene of the target cell, and both; and wherein the chimeric polypeptide retains an essential function of the essential gene in the target cell; and (c) expressing the chimeric polypeptide in the plurality of transgenic cells.

In some embodiments, the method further comprises a step proceeding step (c), comprising determining the expression level of the polypeptide of interest of the chimeric polypeptide in the plurality of transgenic cells.

In some embodiments, the determining comprises determining the mRNA level, the protein level, or both, of the polypeptide of interest, the essential gene, or both.

In some embodiments, the determining comprises determining the mRNA level, the protein level, or both, of the chimeric polypeptide.

In some embodiments, the determining is in a sample or a biological sample comprising at least one transgenic cell of the plurality of transgenic cells.

In some embodiments, the determining comprises at least once determining. In some embodiments, the determining comprises multiple determining.

Methods of gene expression quantification are common and would be apparent to one of ordinary skill in the art. Non-limiting examples of such methods include, but are not limited to, RT-PCR, real-time RT-PCR, western blot, dot blot, densitometry, or others, which would be apparent to one of ordinary skill in the art of molecular biology and cell biology.

### Computer Program

According to some embodiments, there is provided a computer program product, comprising a non-transitory computer-readable storage medium having program code embodied thereon, the program code executable by at least one hardware processor to: (a) receiving a nucleic acid sequence comprising a coding region encoding a polypeptide; (b) selecting a nucleic acid sequence encoding an essential gene of a target cell, and optionally a sequence encoding a linker; and (c) generating a coding sequence comprising the nucleic acid sequence received in (a) and the nucleic acid sequence selected in (b), optionally comprising the sequence encoding a linker between them, wherein the generated coding sequence encodes a chimeric polypeptide that retains an essential function of the essential gene in the target cell.

According to some embodiments, there is provided a computer program product, comprising a non-transitory computer-readable storage medium having program code embodied thereon, the program code executable by at least one hardware processor to: (a) receiving a nucleic acid sequence comprising a coding region encoding a polypeptide; and (b) generating a coding sequence encoding a chimeric polypeptide comprising the polypeptide and an essential gene of a target cell optimized thereto for the generation of a chimeric polypeptide in the target cell, wherein the optimized comprises: a modified GC content, at least one less mutation hotspot, an modified codon usage for optimized expression of the coding sequence in the target cell, at least one less epigenetic hotspot, or any combination thereof, compared to a wildtype nucleic acid sequence encoding any one of: the polypeptide, the essential gene of the target cell, and both; and wherein the chimeric polypeptide retains an essential function of the essential gene in the target cell.

According to some embodiments, there is provided a system comprising at least one hardware processor; and a non-transitory computer-readable storage medium having stored thereon program instructions, the program instructions executable by the at least one hardware processor to: (a) receive a nucleic acid sequence comprising a coding region encoding a polypeptide; and (b) generate a coding sequence encoding a chimeric polypeptide comprising the polypeptide and an essential gene of a target cell optimized thereto for the generation of a chimeric polypeptide in the target cell, wherein the optimized comprises: a modified GC content, at least one less mutation hotspot, an modified codon usage for optimized expression of the coding sequence in the target cell, at least one less epigenetic hotspot, or any combination thereof, compared to a wildtype nucleic acid sequence encoding any one of: the polypeptide, the essential gene of the target cell, and both; and wherein the chimeric polypeptide retains an essential function of the essential gene in the target cell.

In some embodiments, the selecting further comprises selecting a sequence encoding a linker being concatenated to the polypeptide and to the essential gene of the target cell, of the chimeric polypeptide.

In some embodiments, the encoded chimeric polypeptide retains all essential functions of the essential gene in the target cell. In some embodiments, the polypeptide is a target polypeptide. In some embodiments, the polypeptide is the target protein. In some embodiments, the generated coding sequence is a nucleic acid molecule of the invention. In some embodiments, the generating further comprises generating an expression vector comprising the coding region. In some embodiments, the generating further comprises selecting a regulatory element for expression of the coding region.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

In some embodiments, computer program of the present invention comprises Labview or MATLAB.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting of”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Experimental Outline

The inventors create a user-friendly software for the biotechnology industries and synthetic biology labs. This program greatly eases the struggle of expressing, for a long duration, a high level of a target gene or genes, whether it is for therapeutics or for bioproduction. For the creation of this robust and reliable method, the inventors execute empiric experiments and create mathematical models that simultaneously achieve two goals. The inventors prove (both theoretically and empirically) that indeed N-terminally attaching a target gene to an essential gene of an organism greatly increases the evolutionary stability of that target gene while still maintaining high levels of expression at all times, in that organism. The inventors characterize what features of the essential gene are vital for creating a durable genetic construct and what specific features are more important to each individual target gene. This enables the software to customize a specific and suited construct for every target gene.

The inventors compiled a list of 64 features from various databases, and then characterized all of the essential genes of S. cerevisiae by those features. By using a previously generated library (Yofe et al., 2016), the inventors perform an in-lab evolution experiment in a genome-wide manner, measuring the evolutionary half-life of red fluorescent protein (RFP) or green fluorescent protein (GFP) under the same promoter when fused each time to a different open reading frame (ORF). The inventors perform a similar experiment for measuring the evolutionary half-life of strains carrying RFP/GFP under the same promoter when not fused to any gene (hereafter termed “negative control”). Those results simultaneously, validate the inventors' hypothesis and provide insights into which features in combination are important for the evolutionary half-life of the particular construct. As two different target genes are used in this experiment, the inventors are able to extract what features of the essential gene are important when constructed with different target genes. With this data and by using bioinformatics and systematic approach the inventors are able to compose a bigger picture thereby enabling a software to choose the best match of essential-target gene available and thereafter design the optimal fusion of the two genes. At this point, the inventors use the software to create such an evolutionary stabilizing construct for 10 target genes which are wildly used in industries around the world. After building the constructs the inventors test them in an in-lab evolution experiment vs. the version used nowadays in factories and labs around the world.

### Evolution Experiment

One of the great benefits of using S. cerevisiae is the abundance of existing genomic data, strains and libraries. One of such available library is a library of all the ORF in S. cerevisiae fused with either RFP or GFP in the N-′ or C′-terminus. This library initially created for global characterization of protein to protein interaction and protein localization gathered an immense amount of data on each of the essential proteins in S. cerevisiae that assisted the inventors in forming the herein disclosed models. More than that, by using the library and the strains mentioned therein, the inventors were able to devise easy, fast, robust and cheap evolution experiments to validate their hypothesis and gather further data for the software. The inventors decided to use two libraries, one where GFP under a mild consecutive promoter (Nop1) is fused to every ORF. And second is RFP under strong consecutive promoter (Tef2c) is fused to every ORF. Using the libraries, the inventors then created a co-culture system, containing all the different strains, each containing a different ORF attached to a fluorescent protein. One co-culture for each library, and every co-culture in 9 independent repeats. Those co-cultures (along-side the negative control) are grown in Chi.Bio devices for long in-lab evolution experiments. The Chi.Bio device constantly checks the OD₆₀₀ of the cells (as a parameter of growth and density) and the fluorescence of the cells (the intensity of fluorescence indicates toward the expression of the target gene in this experiment). Every time the OD is between 1-1.2, the Chi.Bio device automatically dilutes the media to reach an OD of 0.3, thus keeping the yeast cells always in the logarithmic phase of growth. During the evolution experiment, at different time slots, the co-culture is run through a fluorescence activated cell sorting (FACS), dividing the population to different fluorescence levels. At each such time point, the different populations are sent for deep sequencing to characterize the heterogenicity of each population. The data obtained at every time point is gathered so as to compile a genome-wide characterization of the evolutionary half-life of all the ORFs with the target genes. The inventors also sequence the target gene and its promoter, thus characterizing the different possibilities of a mutation for each essential gene in a construct in a genome-wide manner. Thus, in one experiment, the inventors gather immense data linking between different features of the genes and the evolutionary stability of the construct and compiling a mutational profile of all the different ORFs and more importantly, a mutational avoidance profile (e.g., which mutation is less prone to happen than in the negative control when attached to a gene(s) with a certain feature(s)).

### Software

The main goal of the herein disclosed software is when given a target gene is to provide an output of the best-suited essential gene to pair with, in order to prolong the evolutionary half-life of that target gene. The first stage is to figure which mutations are most prone to happen in that specific target gene, creating a specific mutation profile. The software takes into account: mutational hotspots, the mutational footprint and codon-bias of that organism, the three-dimensional structure of the protein, where and what are the structural vulnerability spots of the protein and what type of mutations can disrupt its structure, and what can disrupt the protein's high expression level. This complied mutation profile is crossed against all the essential genes features and the data on their mutational profile gathered in the in-lab evolution experiment. The different essential gene's features are scored according to two things: (1) the alignment of the mutational avoidance profile of the different features and the target gene mutational profile; and (2) scoring the features important for evolutionary stability for the specific features of that target genes. At the same time, the target protein structure is checked against the structure (predicted or known) of the highest scoring essential genes. Every essential gene is scored by the negative chance that it or the target gene is misfold if fused together, and by the chance that when fused they interfere with each other's activity (e.g., catalytic activity). The software also adds to the score the probability that a mutation inducing the misfolding of the target gene further induces the misfolding of the essential gene. The total score is calculated, and the best scoring genes are provided as an output. The software also optimizes the coding regions of the two genes for optimal performance after fusion.

### Separating the Protein

For further usage of this method for therapeutics or bioproduction, a way to separate the proteins has to be created. At this time there are 4 different possibilities, each suited for a different problem and industry, as delineated herein below.

(1) Linker—Can be wildly used in bioproduction. With a linker, the two proteins stay fused. Linkers can be used in any case wherein the target protein is not purified on its own and/or that the fusing of the protein does not affect the activity of the target protein (which is predictable using the herein disclosed software).

(2) 2A-2A peptides are 18-22 amino-acid long viral oligopeptides that mediate “cleavage” of polypeptides during translation in eukaryotic cells. This system has been used before by biotechnology companies, and therefore is easy to implement. It is an easy solution if the fusing of the target protein in some way hinders its catalytic activity. However, the cleavage yields a ‘left-over’ of between 20-22 aa long (dependents on the kind of 2A used) in the N′ terminus of the protein. This may be a drawback in cases wherein the purpose is to manufacture and/or purify the protein itself.

(3) Pseudoknots—Ribosomes typically translate mRNA without shifting the translational reading frame. However, several organisms have evolved mechanisms to cause site-specific or programmed frameshifting of the ribosome in either the +1 or −1 direction. This ribosomal frameshift is facilitated by RNA structures known as “pseudoknots”. The frameshift happens in a fixed frequency or rate (per each pseudoknot it may be a different frequency or rate, but on average, per all of them it is about 1-10%) and it correlates with the strength of the pseudoknot structure. This system can be applied to express two versions of the protein with one DNA sequence. In the main transitional frame, only the target protein is translated, possibly with a leader sequence, allowing for extracellular extraction, and in the other frame, the target and the essential protein are translated as a fused protein. By applying this method, the expression level of the target gene is greatly upregulated, and more importantly a purified extracellular protein free of any unwanted left-over from the cleavage, is obtained, thus making this system appealing for therapeutics and pharmaceutical companies.

(4) Protease—By using a protease (in-vitro or in-vivo) the two proteins (e.g., the target and the essential protein) can be easily separated without any side effect or left-over. While highly appealing, the site-specificity and the low number of known and wildly used proteases are quite limiting, and thus may work in a relatively limited number of cases.

### Constructing and Testing the Gene Circuit

After receiving the best match for a target gene generated by the herein disclosed software, the genetic circuit is constructed and examined by an in-lab evolution experiment. An oligonucleotide containing a strong promoter, and the target gene fused (with the cleaving system in place) to its compatible essential gene, is ordered. This oligonucleotide is inserted into a plasmid backbone with a URA selection marker. After transforming the plasmid to the cells the chosen essential gene is deleted from the genome (so the only copy is the one located on the plasmid) with a Kanamycin resistance selection marker with flanks of the genomic essential gene (so as to erase only it and not the copy located on the plasmid). Then plating the cells on URA⁻ plates and 5 fluoroorotic acid (5FOA) plates (which allow for counter selection of URA⁺ cells, so only cells that have lost the plasmid are able to grow). If the cells grow on the URA⁻ plates and not growing at all on the 5FOA plates, it is concluded that the construct works. Then the next step is performing an evolution experiment as previously described while checking at different time points for the presence, expression level, and the accumulation of a mutation, using western blot and sequencing.

### Proof-of-Concept Experiment

The evolutionary half-life of ten diverse constructs was compared against the half-life of the unattached target gene (GFP alone as the negative control for stability). Each of the ten tested constructs is derived from the GFP SWAT library Yofe et al., 2016. Thus, it is composed of GFP, a fixed linker, and an essential gene. The 10 constructs were selected based on a hierarchical clustering algorithm and many descriptive features the inventors created to characterize the genes and define their “diversity”.

### Genes Selection

The inventors address the 10 genes selection as a classification problem with an unknown number of clusters and utilized the K-means algorithm as described in MacQueen, (1967) with 636 genes, 336 dimensions when every dimension represents one feature.

K-means tends to converge to local minima and is often biased by the determination of initial centroids. Thus, the inventors used multiple runs (n=1,000) and made sure that the chosen centroids in each iteration are frequent. Another requirement of the algorithm is to specify in advance the number of clusters, which depends on the data distribution. The inventors used two methods to overcome this problem: (a) based on the Euclidean distance metric, the inventors can describe the k-means algorithm as a simple optimization problem, an iterative approach for minimizing the within-cluster Sum of Squared Errors (SSE), which is sometimes also called cluster inertia:

$$SSE = {\sum }_{i = 1}^{n} {\sum }_{j = 1}^{k} {w}_{i , j} {\vert {x}_{i} - {μ}_{j} \vert}^{2} , \qquad eq . 1$$

where Xᵢ is the i^{th} observation (single data point/sample), μⱼ is the centroid for cluster j, and w_{i,j} is the weight: it is equal to 1 where the sample Xᵢ is in cluster j and otherwise it is set to 0. Inertia can be recognized as a measure of how internally coherent clusters are, under the assumption that clusters are convex and isotropic, which is not always the case. This assumption sometimes leads to poor response to elongated clusters or manifolds with irregular shapes, but since the inventors are not concentrating on too many clusters it is sufficient and helps us simplify the problem from an engineering perspective. Using this method to find the elbow in the graph of SSE for each K will lead to the optimal K; (b) The inventors used the silhouette criteria:

$$s ( i ) = \frac{b ( i ) - a ( i )}{max \{ a ( i ) , b ( i ) \}} \qquad eq . 2$$

for each data-point i, a(i) is the average dissimilarity of i with all data-points in the cluster of i; b(i) is the average dissimilarity of i with data-points not in the cluster of i. The inventors then run the herein disclosed K-means algorithm with K=1, . . . , 150 while analyzing the values of both SSE and Silhouette methods for every tested K. The optimal K for the herein disclosed data was K=133. The inventors narrowed it down to the 10 most diverse centroids by analyzing only the K that showed maximal Silhouette values. This resulted in a list of 8 values, of which the smallest is 15 and the largest is 133. The inventors then ran the K-means algorithm again 8 times, one for every K from the list. Every iteration provided K centroids, from which the inventors saved only the ones that repeated in all the iterations. This process resulted on exactly 10 centroids. The genes closest to the centroid were selected as they best represent the cluster.

### Strains and Growth Conditions

The N′ SWAp Tag (SWAT)-GFP library strains derived from S. cerevisiae BY4741 background strain (MATa, his3Δ1, leu2Δ0, met15Δ0, ura3Δ0) (Baker Brachmann et al., 1998. The N′ SWAp Tag contains a URA3 as positive selection marker and a constitutive promoter SpNOP1pr which confers medium-level expression Yofe et al., 2016. The specific strains that were chosen for this experiment are listed in Table 3.

TABLE 3
Ten selected diverse genes for the proof-of-concept experiment.
Systemic name and description are derived from the
Saccharomyces Genome Database (SGD). The
source of all strains is the SWAT library.

| | Gene systemic name | Genotype |
|---|---|---|
| 1 | YKL019W | GFP-ram2 |
| 2 | YGL130W | GFP-ceg1 |
| 3 | YOR236W | GFP-dfr1 |
| 4 | YMR113W | GFP-fol3 |
| 5 | YLR347C | GFP-kap95 |
| 6 | YDL193W | GFP-nus1 |
| 7 | YNL272C | GFP-sec2 |
| 8 | YIR012W | GFP-sqt1 |
| 9 | YKL019W | GFP-tsc13 |
| 10 | YDL164C | GFP-cdc9 |

The grew triplicates of each construct separately, with unattached GFP construct as negative control for stability. Every day, the inventors repeatedly transferred them to fresh media for overnight growth, measuring fluorescence and optical density (OD) for approximately 180 generations. The evolution experiment took place in 96-Deepwell Plates (Starlab group Cat. No. S1896-2110) with each well containing 450 μl of Synthetic Defined (SD)-complete liquid media and incubated over night at 30° C. Each day, 50 μl of saturated yeasts was diluted in 450 μl sterile double distilled water (DDW) pipetted and transferred 50 μl to a new plate. Correspondingly, a copy of the plate was transferred each day to a sterile 96-Deepwell plate with 200 μl Synthetic Defined (SD)-URA as negative control detecting contaminations. Every couple of days, samples were frozen stocked in 40% glycerol at −80° C. for backup. The unattached GFP with the same promoter SpNOP1pr transformed to the background strain BY4741 using the transformation protocols as described by Gietz, R. D. and R. A. Woods (2002).

### Quantification of GFP Fluorescence Intensity

Every five days 200 μl of samples transferred to a new plate and centrifuged for 1 minute at 4,000 rpm. Supernatant discarded and the pellet pipetted with 200 μl of 10× diluted PBS. Samples were treated that way twice to get rid of YPD remnants before quantified. 100 μl of samples was transferred to Thermo Scientific™ 96 Well Black/Clear Bottom Plate for quantification. Cell density was determined using spectrophotometry (OD₆₀₀) and florescence (excitation wavelength 490 nm, emission wavelength 535 nm, excitation and emission bandwidth 20 nm, gain set to manual 100) using Spark® Multimode Microplate Reader by Tecan.

### Feature Generation for Co-Stability Prediction

The analyzed genes for the co-stability prediction models are 6,726 yeast's ORF retrieved from the Saccharomyces Genome Database (SGD; http://www.yeastgenome.org/). Features were calculated for all genes unless stated otherwise in the supplementary data (for example, codon usage bias was calculated only on genes divisible by 3). In total, 1,962 features were calculated. Herein below are several examples.

### Protein to Protein Interaction Networks and Graphs

Essential proteins are those that are indispensable to cellular survival and development and are usually more stable. Existing methods for essential protein identification generally rely on knock-out experiments and/or the relative density of their interactions (edges) with other proteins in a Protein-Protein Interaction (PPI) network, as described in Wang et al., 2014. The idea is that genes that create a larger number of interactions are probably evolutionarily conserved. In addition, the more active segments a gene has, the more functional the gene is, thus, will tend to develop fewer mutation. For these reasons there may be a high correlation between high rank and high levels of gene expression. The inventors used 4 different data sources of PPI networks (STRING, Szklarczyk et al., 2019; TheCellMap, Usaj et al., 2017; YeastMine, Balakrishnan et al., 2012; and BioGRID, Oughtred et al., 2019) and created many features which relate to the amount of interactions that a gene has and to the type of interactions.

### Transcription Factors

The process of transcription is the first stage of gene expression, resulting in the production of a primary RNA transcript from the DNA of a particular gene. Both basal transcription and its regulation are dependent upon specific protein factors known as transcription factors. These factors bind to specific DNA sequences in gene regulatory regions and control their transcription, as described by Latchman, 1993. The number of transcription regulatory sites is an important feature because the transcription factors regulate the expression levels of the genes, and thus can help to predict the intensity of fluorescence that the inventors are interested in. The inventors used YEASTRACT database (Teixeira et al., 2018) to extract this feature.

### Codon Usage Bias

Often, genes that are highly biased in terms of their codon usage are regulated by the cell or evolution to maintain specific expression values or a unique function. Thus, they are hypothesized to include preserved areas, as they are biased towards signals that promote (or inhibit) their translation or stability. The features included protein abundance, effective number of codons (ENC) (Wright, 1990), Codon Adaptation Index (CAI) (Sharp and Li, 1987), Relative Codon Adaptation (RCA) index (Fox and Erill, 2010), tRNA Adaptation Index (tAI) (Tuller et al., 2010), Normalized Translation Efficiency (NTE) index (Pechmann and Frydman, 2013), and many more.

Local Folding Energy (LFE) of mRNA

Folding of mRNA affects the efficiency of gene expression. The stronger the fold, the more energy must be expended to release the fold and it will be more difficult to perform the translation process. This can impair the level of expression of the gene. the inventors used LFE information data (Peeri and Tuller, 2020) and created features based on the LFE of genes in varying sliding windows.

### Target Gene Related Features

For each analyzed gene the inventors have calculated profiles of codon relative frequency, amino acid frequency, GC content etc., using sliding windows of specified size. Several distance metrics were applied between each profile and the target gene's profile. If the target gene and an essential gene exhibit similar profiles (small distances), then they are likely to be functionally or structurally similar. Thus, they could create more stable constructs. The inventors have also generated the CAI and RCA features with the target gene as a reference. These features will estimate for each gene its extent of bias toward codons (CAI) or nucleotides (RCA) that are known to be favoured in the target gene.

### Correlation-Based Feature Selection for Co-Stability Prediction

ORFs that belonged to noncoding or paralog genes (according to YeastMine, Balakrishnan et al., 2012) were removed from the analysis, being either poorly characterized or irrelevant to the study's end goal (not contributing to the co-stability). A threshold was set such that genes with more than 10% missing features are eliminated from the analysis. Next, features that expressed more than 600 missing values were removed.

This pre-processing resulted in 1,949 features and 4,035 instances. Then, a heuristic feature selection was implemented, based on the Filter paradigm (Kira and Rendell, 1992; and Almuallim and Dietterich, 1994) which operates independently of any learning algorithm. The feature with the highest Spearmen correlation served as the starting point for the Sequential Forward Selection (SFS) search algorithm (Kira and Rendell, 1992). The evaluation merit in each iteration was defined as CFS (correlation-based feature selector) measure (Hall, 1999):

$${M}_{s} = \frac{k {\underline{r}}_{cf}}{\sqrt{k + k ( k - 1 ) {\underline{r}}_{ff}}} , \qquad eq . 3$$

where Mₛ is the heuristic merit of a feature subset S containing k features, r_{cf} is the mean feature-label correlation, and r_{ff} is the mean feature-feature correlation. This way, 300 features were selected.

### Co-Stability Predictor Development

The co-stability predictor is a machine-learning linear regressor. The predicted values for training and testing of the model are median intensity (fluorescence) of all strains in the SWAT library (yeast strains, where each variant has a fluorescent gene, GFP or RFP, fused to its N terminus). The values are derived from SWAT database (Yofe et al., 2016; and Weill et al., 2018), and include two GFP sets: (1) NOP1: Median of GFP intensity of all strains tagged with GFP under the synthetic promoter NOP1; (2) Native: Median of GFP intensity of SWAT strains under their native promoter, after swap with Seamless GFP donor. As a test set, the inventors used two RFP sets: (1) TEF2pr-mCherry: Median of mCherry intensity of SWAT strains after swap with Tef2-mCherry donor; (2) Tef2pr-VC: Median of GFP intensity of SWAT strains after swap with Tef2-VC donor.

The objective of the model is to achieve accurate prediction of top 1% of the genes (with the highest fluorescent protein expression). For this purpose, the inventors suggest two methods to predict the fluorescent protein expression as a normal regression problem and suggest an innovative evaluation method.

### Prediction Methods

The first tested method is a regression using an artificial neural network (ANN). The inventors designed a fully connected ANN with 5 layers: an input layer, 3 hidden layers, and an output layer. The simplest model uses normalized and pre-selected features as input. The size of the hidden layers is the input size divided by 2, 4 and 8 respectively, and the output layer size is 1 as the inventors are computing a single value as an output. The herein disclosed network uses the leakyRelu (Wang et al., 2015) activation function and trained with several methods of the loss function to obtain the best prediction. While training, the inventors apply several methods of regularization, as dropout (Srivastava et al., 2014) and batch normalization (Ioffe and Szegedy, 2015).

The second method the inventors tried is regression using the random forest (RF) algorithm (Breiman et al., 1984). RF is a supervised machine learning algorithm which uses many decision trees. It is a way of averaging multiple decision trees, which are trained on different parts of the same training set, to reduce the variance. The decision trees are called “estimators”. The number of estimators determines the model's size. To select the best model size, the inventors scanned various numbers of estimators to achieve the best RF architecture.

The model was developed only against the GFP databases. In total, four configurations were developed: RF-Native, RF-NOP1, ANN-Native and ANN-NOP1. The inventors refer to GFP as the target gene and the yeast's genes as the candidates for linkage. For this task, the features related to the target gene were calculated according to the GFP sequence.

### Active Feature Selection

After receiving 300 features for every gene from the passive feature selection algorithm, the inventors implemented active feature selection (Wrapper paradigm (Kira and Rendell, 1992; and Almuallim and Dietterich, 1994)) to reduce the number of features and to improve the network accuracy. This feature selection is based on the Lottery Ticket Hypothesis (Frankle and M. Carbin, 2018).

First, the RF model was trained with all the initial features; then, the inventors analyzed the feature's importance and their effect on the herein disclosed results and filtered the least 5% influential features. This process was repeated until the last influential feature is above a certain threshold that was set. This process resulted in 201 features and improved the results significantly.

### Evaluation Metrics

Since only the fluorescence values of the best (most fluorescent) genes need to be predicted for the predictor to be considered efficient, the inventors focused the prediction efforts on the right tail of the fluorescent protein expression distribution. Meaning, the inventors are trying to accurately predict the outliers of the data. To overcome this challenge, the inventors used 3 evaluation metrics to evaluate the herein disclosed model prediction of the best genes: R², accuracy and agreement score.

The R² (or coefficient of determination) is defined as:

$${R}^{2} = 1 - \frac{u}{v} , \qquad eq . 4$$

where u is the residual sum of squares u=Σ(yₜᵣᵤₑ−y_{pred})² (y_{pred} is the predicted value, yₜᵣᵤₑ is the true value) and v=Σ(yₜᵣᵤₑ−ŷₜᵣᵤₑ)² denoting the proportion of variance of yₜᵣᵤₑ that has been explained by the features in the model.

The second evaluation metric the inventors termed accuracy, and define as the number of genes with a prediction error e=|yₜᵣᵤₑ−y_{pred}| within a threshold of 10 au, divided by the size of the test set:

$$Accu racy = \frac{{N}_{N umber of genes with e < TH}}{Size of test set} , \qquad eq . 5$$

The third evaluation metric is the agreement score. As mentioned before, the predictor should predict the genes with the highest fluorescent protein expression, which the predictor should recommend as the best genes to linkage. The agreement score indicates the ability of the predictor to predict which genes are rated as genes with high fluorescent protein expression, without attaching importance to the exact value of the fluorescent protein expression. Given a number N of top best genes (20 in this case), the metric is defined as the number of overlapping genes between the N top best genes in the predicted and ground truth values.

### Linker Selection Module

Two types of linkers were analyzed and suggested in the sTAUbility Enhancer software: 2A and fusion linkers. Regarding the 2A linkers, 4 options of linkers are presented—their names, sequences and scores in descending order of efficiency (Kim et al., 2014), such that the user could take into consideration other factors rather than efficiency.

As for fusion linkers, the included linkers were derived from the linkers' database provided by The Centre for Integrative Bioinformatics VU (IBIVU) containing 1,280 linkers (http://www.ibi.vu.nl/programs/linkerdbwww/) (George and Heringa, 2002). IUPred2A tool (Meszaros et al., 2018) was used in order to compute both the disorder profiles of the essential and target genes before and after the fusion. This tool aims to identify Intrinsically Disordered Protein Regions (IDPRs, i.e., protein segments that have no single well-defined tertiary structure under native conditions) based on a biophysics-based model. It enables the user to input any protein sequence and returns a score between 0 and 1 for each residue, corresponding to the probability of the given residue being part of a disordered region. There are three different disorder prediction types offered by IUPRED2A, each using different parameters optimized for slightly different applications. These are: long disorder, short disorder, and structured domains. The long disorder predicts global structural disorder that encompasses at least 30 consecutive residues of the protein (Meszaros et al., 2018). It was found most useful for the model's task.

Two different scores were calculated based on Euclidean distance for each of the ˜1,300 linkers: (1) Essential (or conjugated) gene's score: a Euclidian distance between the essential gene disorder profile before fusion and after fusion. (2) Target gene's score: a Euclidian distance between the target gene disorder profile before fusion and after fusion. These scores were weighted equally and formed the final score.

final score (L)=0.5·s+0.5·t eq. 6,

where L is the analyzed linker, s is the essential (or conjugated) gene's score, and t is the target gene's score. The ideal linker was the one with minimal final score. The 10 best linkers (from the lowest to highest scores in the 10 best linkers) are presented to the software's user including their sequence, name, score, and a graphic view of the change in their disorder profile (see FIG. 7). In that way, the user is able to consider additional factors relevant for his purposes.

### Optimization Engine

The optimization process provided by the sTAUbility EFM optimizer utilizes the Python package DNA chisel (Zulkower and Rosser, 2019) (version 3.2.3), allowing for optimization of DNA sequences with respect to a set of constraints and objectives.

For any given input sequence, the optimization procedure involves two steps: (a) optimize mRNA folding at the start of sequence, codon usage and required GC content; (b) avoid mutational patterns (SSR, RMD and methylation when relevant) detected by the previously described methods in the semi-optimized sequence, while maintaining the codon usage and GC content as much as possible and not changing the start of the sequence (where the mRNA folding was optimized).

The GC content optimization refers to the maintenance of the frequency of GC nucleotides within a specified range. The algorithm splits the sequence to windows of a specified size (default 1/50 of the sequence length) and optimize within each window. The lower the GC content, the more stable is the sequence, since it has been proved that genes with high GC had a substantially elevated rate of mutations, both single-base substitutions and deletions (Kiktev et al., 2018).

The mRNA folding optimization is implemented at the first 15 codons of the input sequence. At the open reading frame (ORF) 5′ end, there is a well-conserved signal for weak folding of RNA (Tuller and Zur, 2015), as it promotes recognition of the start codon by the pre-initiation ribosomal complex. For the optimization problem, the inventors defined a new constraint that maximizes the local free energy (LFE) of the most folded structure in the first 15 codons. The LFE secondary structure is predicted using the seqfold tool (T=32*), as it is easy to integrate. This tool computes minimum folding energy and corresponding secondary structure from RNA sequences, using the thermodynamic nearest-neighbor approach. The MFE structure of an mRNA sequence is predicted using a loop-based energy model and the dynamic programming algorithm introduced by Zuker and P. Stiegler, 1981, and recently improved by Mathews et al., 2004.

In codon optimization, the algorithm replaces the codons used to generate amino acids, using four different codon usage tables: (1) relative codon frequency within the host organism (Nakamura et al., 2000). The optimization methods are “use best codon”, “match codon usage” and “harmonize RCA”, all described in Zulkower et al., 2019. (2) “tAI”: tRNA Adaptation Index, a biophysical measure of codon usage bias that scores the adaptation of codons to the tRNA pool in yeast (Tuller et al., 2010). The optimization method is “use best codon”. (3) “nTE”: the normalized translation efficiency, a biophysical measure of codon usage bias that scores the codons according to their adaptation to the supply and demand of the tRNA in the yeast (Pechmann and Frydman, 2013). The optimization method is “use best codon”. (4) “TDR”: typical decoding rate. for each codon, this empirical measure is defined as the codon decoding time's reciprocal. The typical decoding time of each codon was taken from this database (S. cerevisiae, Exponential) (Dana and Tuller, 2014; and Dana and Tuller, 2015). Then, it was converted to rate and normalized to the range of 0-1. Since this measure is not defined for stop codons, the weights of these codons are set to their frequency in the host genome. The optimization method is “use best codon”.

The avoidance of mutational hotspots such as simple sequence repeats (SSR) and recombination mediated deletions (RMD) improves genomic stability. For computational considerations, the inventors only offer to avoid the first 10 most probable sites from each type.

### RAID and SSR Sites' Calculation

The original EFM calculator considers three forms of mutation—SSR, RMD and BPS, the latter gives a baseline mutational probability for comparison. From these, a Relative Instability Prediction (RIP) score is calculated as follows:

$$R I P = \frac{S S R + RMS + BPS}{BPS} , \qquad eq . 1$$

This score gives a measure of how unstable a sequence is, where its minimum is 1 for the case of no SSR or RMD mutational hotspots.

The following equations are based on empirical data, collected from previously described art. The data was fitted with a log-linear approximation, providing generational mutation rates for E. coli. These rates are expected to be correlative with highly mutable sites within other organisms.

SSRs are sites composed of a repeating short sequence, causing potential polymerase slippage. For instance, the following sequence is an SSR: (AT)(AT)(AT)(AT); it has a base unit length (L) of 2 and number of units (N) of 4. The calculator considers SSR sites if they have (N≥3, L≥2), or (N≥4, L=1). Marking generational mutation rate as μ, the SSR score of a site is calculated as follows:

$$log {μ}_{S S R} = \{ \begin{matrix}- 12. 90 + 0.729 N , & L = 1 \\ - 4.7 \; 49 + 0.0 \; 63 N , & L > 1\end{matrix} , \qquad eq . 2$$

These rates are based on the empirical data previously collected.

RMDs are long (L≥16), identical sites appearing in different locations in the sequence, causing potential recombination faults. The recombination probability between two sites is based on their length L and the distance between them Lₛ, and is calculated as follows:

$${μ}_{R M D} = {( A + {L}_{s} )}^{- \frac{α}{L}} \cdot \frac{L}{1 + B L} , \qquad eq . 3$$

where A=5.8±0.4, B=1465.6±50.0, α=29.0±0.1 were found empirically.

Different rates were found for B. subtilis (a recA⁻ strain), however it is a less generic model for other organisms.

BPS is the probability of spontaneous mutation. It was empirically estimated from genome sequencing of E. coli mutation accumulation lines, as μ_{BPS}=2.2·10⁻¹⁰.

Note that all empirical findings were estimated for E. coli. While the probability of mutation will be different for other organisms, the ranking of hypermutable sites is approximately maintained.

### Methylation Sites Calculation

As previously stated, the epigenetic inheritance process of methylation has a much more dominant effect on activation and expression within mammalian and insectoid cells (e.g., Chinese hamster ovary, CHO cells). In the following analysis, the inventors provide a method for detection of highly probable methylation sites.

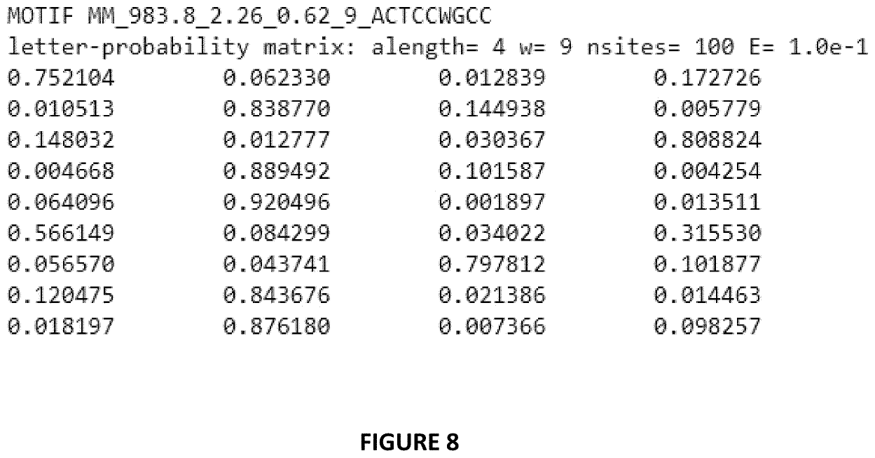

In a study published by Wang et al., 313 methylation motifs are identified and analyzed in somatic, brain, liver and pancreas cells. A motif is a sequence pattern that occurs repeatedly in a group of related sequences. The MEME (Multiple Expectation maximizations for Motif Elicitation) Suite is a collection of tools for the discovery and analysis of sequence motifs, within which motifs are represented as position-dependent nucleotide probability matrices, that describe the probability of each nucleotide at each position in the pattern. The reported motifs in Wang's database (http://wanglab.ucsd.edu/star/MethylMotifs/) are presented in MEME minimal format (FIG. 8).

This database details, per methylation site, what is the likelihood of receiving a nucleotide sequence. This is commonly called a Position Probability Matrix (PPM). By normalizing each probability with the nucleotide background probability in the database's host organism and taking the logarithm of these values, a Position-Specific Scoring Matrix (PSSM) is generated. This scoring mechanism is a common method in the field for scoring likelihood of various sites.

For each genetic subsequence, the following calculation from Bayesian statistics provides us with the theoretical basis for estimating its methylation site and how likely is it:

$$p ( tested methylation site \vert sequence ) = \frac{p ( tested methylation site )}{p ( sequence )} \cdot p ( sequence \vert tested methylation site ) \qquad eq . 4$$

$$\frac{p ( sequence \vert tested methylation site )}{p ( sequence )}$$

is inherently described by the PSSM score, and p(tested methylation site) is assumed to be uniform. Thus, the site and likelihood can be estimated by finding the site maximizing the PSSM score. A score higher than 0 indicates a higher probability of being a methylation site than a randomly generated site, and the higher the score, the more likely. Thus, the inventors can find the most likely sites within a sequence and rank them, finding the sites most in need of deletion or editing. The inventors note that this database is highly comprehensive and permits an evaluation of p(sequence|tested methylation site) with a high level of detail. However, it does not give an estimation of the likelihood or strength of the methylation process.

Users may choose to use alternative PSSM matrices, in MEME minimal format as well. This standard format allows users to import custom motifs from alternative sources, allowing avoidance of sites dictated by individual engineering needs, other than methylation.

### Optimization Engine

The optimization process provided by the ESO utilizes the Python package DNA chisel (https://github.com/Edinburgh-Genome-Foundry/DnaChisell) (version 3.2.3), allowing for optimization of DNA sequences divisible by 3 with respect to a set of constraints and objectives. The following constraints are implemented: Enforce Translation (match the target amino acid translation), Enforce GC content (in windows of 1/50 of the sequence length) and Avoid Pattern (for the mutational hotspots detected). The objective is Codon Optimization based on usage table provided by python-codon-tables package (https://pypi.org/project/python-codon-tables/), for available organisms only: B. subtilis, C. elegans, D. melanogaster, E. coli, G. gallus, H. sapiens, M musculus, M musculus domesticus, S. cerevisiae. For computational considerations, the inventors only offer to avoid the first 10 most probable sites from each type (SSR, RMD and methylation or custom motif).

### Conservation Score Analysis

Utilizing the ConSurf program, a conservation score was calculated for each nucleotide in different genes of Saccharomyces cerevisiae: Pol30, Pol3, Cdc9, Tor2, Mgs1, Sec9, Glg2, Rnh1, Gnd1, Rga2, Arh1, Yuh1, Siz1, Sir3 and Rad52. These proteins were also analyzed using ESO to mark areas that are predicted to be evolutionary unstable.

The ConSurf tool creates multiple sequence alignment (MSA) out of all the orthologous genes of 100+ organisms for a given sequence. Then, it sums the appearance of each nucleotide in a specific position across all the orthologues genes. A well-conserved nucleotide will appear across all the different orthologs genes, while an evolutionary unstable nucleotide will appear significantly less. The number of times each of the nucleotides appear across the MSA is counted and calculated so that every nucleotide is given a conservation score.

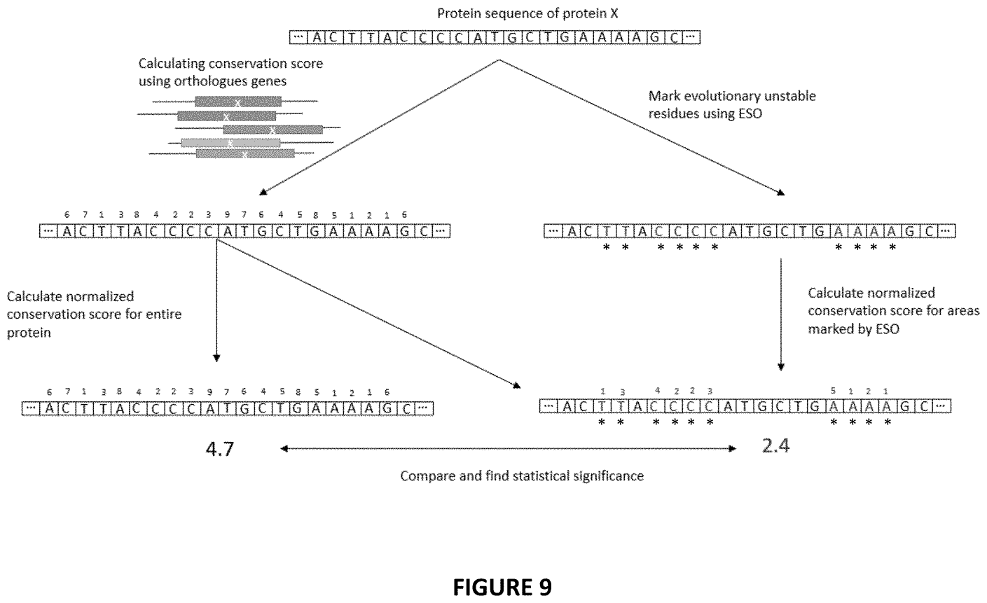

Using this tool, the average conservation score of each protein was calculated and compered to the average conservation score of areas indicated by the ESO to be evolutionary unstable in the same protein. The entire process is illustrated in FIG. 9.

### Gene-Seq Protocol

Creating genetic library.

In lab evaluation experiment.

DNA extraction.

Overnight resection using enzymes chosen according to the given gene and the used ORF (condition such as temperature and concentration of the resection reaction should be conducted as recommend by NEB for the chosen enzyme).

Over-nigh ligation with T4 ligase in 16° C., 50 ng of DNA for each 20 μl reaction. Preform several reactions instead of increasing the volume of the reaction.

PCR reaction (condition will vary depending on the chosen primer, it is recommended to do between 40-50 cycles of amplification).

Repeat step 5 (e.g., ligation).

Preformed RCA reaction according to the instruction on the commercial kit.

Sequence in Nano-pore machine.

Computational analysis of the sequencing results for estimating the mutation rate in each variant.

### Example 1

### A Proof-of-Concept Experiment

In this study, the inventors proposed interlocking a desired target construct upstream to a stabilizing essential or fitness-reducing gene in the host organism. The inventors hypothesized that by interlocking a target gene to different conjugated genes their mutational stability could be increased significantly, by varying degrees. To validate this assumption, a proof-of-concept experiment was designed in which the evolutionary half-life of ten diverse constructs was compared against the half-life of the unattached target gene.

Using the Green fluorescent protein (GFP) as a target gene, the inventors defined an indicative stability measure that reflects how much fluorescence was kept in the last day of the experiment compared to the initial fluorescence. This measure ranges from 0-1, wherein 1 means that the fluorescence did not change during the experiment (e.g., the construct is stable), and 0.5 means that half of the initial fluorescence was kept during the evolution experiment.

The fluorescence readings after 180 generations, relative to the experiment's initial fluorescence are displayed in FIG. 3. The negative control (‘GFP alone’) had the second-lowest normalized fluorescence at the end of the experiment, and there was a significant increase in the evolutionary stability of GFP when fused to an essential gene. Moreover, there was a significant difference in the ability of different conjugated genes to evolutionary stabilize the GFP.

The results demonstrate two properties: (1) The target-essential gene linkage significantly prolongs the target gene's evolutionary half-life in the vast majority of cases; and (2) Different essential genes provide varying stability levels, emphasizing the need for careful selection of the host gene to be included in the combined construct, which the herein disclosed software can provide.

However, different genes cause a different amount of metabolic load on the host cell (Glick, 1995). Therefore, the initial fluorescence level of each strain is different, depending on the conjugated gene. This could undermine the reliability of the results, as the difference in the fluorescence kept over the experiment might not correlate with the conjugated gene, but rather to the initial fluorescence level of the strain. Thus, to further increase the credibility of the results, the inventors calculated the Spearman correlation between the initial fluorescence and the maintained normalized fluorescence at the end of the experiment. The results show no significant correlation (FIG. 4).

### Example 2

### Co-Stability Prediction

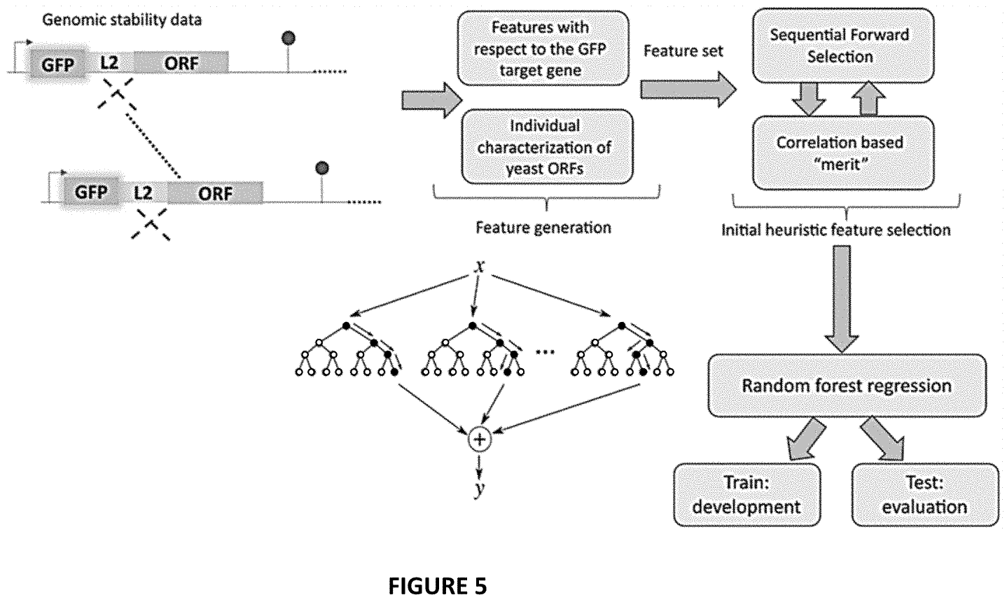

The inventors devised an innovative machine-learning prediction model for genetic co-stability, e.g., the stability of two genes interlocked together, using bioinformatic tools and empirical data collected from large-scale genomic experiments. The model development process is described in FIG. 5.

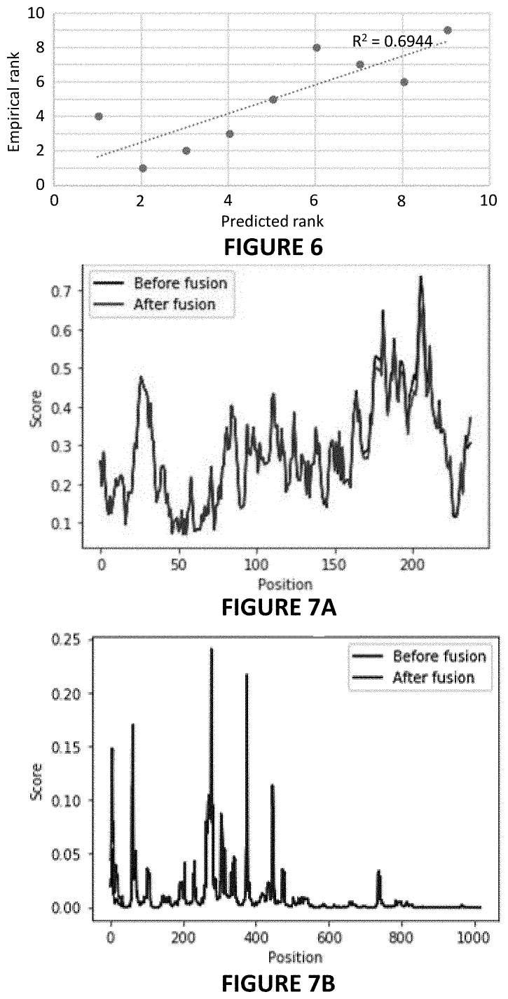

The data used to train the model included fluorescence measurements of all the open reading frames in yeast when attached to GFP as a target gene, derived from SWAT database (Yofe et al., 2016; and Weill et al., 2018). The use of fluorescence levels as stability measurement is justified later in this section (see FIG. 6).

A main challenge of this study was that the stability of genes is poorly characterized. Since the inventors do not know in advance which factors cause gene to be more or less genetically stable, they have generated many descriptive features for each of the yeast's open reading frame (ORF), utilizing various approaches such as bioinformatic analysis, academic publications, empirical measurements etc. Some of the features were calculated considering the gene alone, and some are features with respect to the target gene as well, since the aim is to predict the co-stability and find the best fitting gene to the linkage. This process resulted in 1,962 features. To avoid overfitting and reduce the model's dimensionality, a smaller subset has been selected utilizing a heuristic correlation-based approach (Hall, 1999). This small set is composed of 300 features that are highly correlated to the labels (the predicted values) but less correlate with one another. It appeared that the most significant features, which achieved the highest merit, could be divided into three groups: (a) protein subcellular localization status under various stress condition, derived from LoQAtE database (Breker et al., 2013; and Breker et al., 2014); (b) factors related to transcription mechanism (such as mRNA abundance, bio-physical codon usage indices, and protein abundance); and (c) protein to protein interactions. This result emphasizes the mutual connection between genomic stability and the expression mechanism of the gene, which is why both issues are considered in the optimization process provided by our software.

The predictor performance on a test set was evaluated in four configurations: ANN-NOP1, ANN-Native, RF-NOP1 and RF-Native (see Material and Methods). Three different metrics were used for the assessment, described in the Methods section. These metrics were designed specifically to fit the study's goal: to rank the available yeast's genes according to their co-stability when linked to a given target gene. Thus, metrics such as R-Square are less relevant (although presented) because they do not acknowledge the ranking agreement between the prediction and the true values.

The results in Table 1 clearly demonstrate that both of the RF models (RF-NOP1, RF-Native) are much more accurate and efficient when tested on the GFP test set. This is probably due to the RF's unique “wisdom of the crowd” decision-making approach, which usually results in much more stable models when compared to ANNs.

TABLE 1
Co-stability predictor performance on the GFP test set in the
four tested configurations.

| | | | Median | Agreement on 20 |
|---|---|---|---|---|
| Configuration | R² | Accuracy | error | leading candidates |
| ANN-NOP1 | −0.469 | 0.232 | 24.126 | 11 (0.55) |
| ANN-Native | −1.841 | 0.498 | 10.076 | 7 (0.35) |
| RF-NOP1 | 0.5499 | 0.256 | 23.183 | 11 (0.55) |
| RF-Native | 0.463 | 0.641 | 5.638 | 10 (0.5) |

ANN = artificial neural network.
RF = random forest.
NOP1 = GFP intensity of the strains under synthetic promoter.
Native = GFP intensity of the strains under native promoter.

Additionally, in order to choose between the RF models, the inventors tested the generalization of each model for: (1) the other task (for example, the inventors used the RF-NOP1 model to predict the Native database); and (2) a new target gene using fluorescence measurements of ˜4,000 genes in yeast when RFP is attached to their N′ terminal (Yofe et al., 2016; and Weill et al., 2018) (see Material and Methods for description of the database). The agreement on 20 leading candidates was calculated for each assessment. Although the results in Table 2 are lower than those on GFP, they still prove that the inventors can utilize, to some extent, the GFP-trained model to predict the RFP results.

TABLE 2
Co-stability predictor performance evaluation on the GFP and RFP
test sets, to assess generalization of each RF model. The metric
used in the evaluation is agreement on 20 leading candidates.

| | | | RFP | RFP TEF2pr- |
|---|---|---|---|---|
| Configuration | NOP1 | Native | TEF2pr-VC | mCherry |
| RF-NOP1 | — | 6 (0.3) | 4 (0.2) | 5 (0.25) |
| RF-Native | 6 (0.3) | — | 3 (0.15) | 4 (0.2) |

Finally, the inventors decided to work with the RF-NOP1 model for 2 reasons. First, considering both the GFP and RFP test sets, the results of the RF-NOP1 were a little better than the RF-Native based on both R² (for GFP) and agreement metrics. Second, there is a biological preference for selecting the NOP1-trained model over the NATIVE-trained. In future versions of the herein disclosed software, the inventors would like to propose synthetic promoters for increasing the stability and expression levels of the given construct. Theoretically, the inventors may expect a native and a synthetic promoter to return similar rankings of genes that improve stability. That is, genes predicted to be highly stable, will present high levels of stability in the second model. However, in reality, the behavior of constructs under a synthetic promoter is less predictable. The use of synthetic promoters adds a degree of stochasticity to the system. A promoter replacement can cause some of the genes to be in over-expression or under-expression. The inventors do not know in advance how the genes behave in such a situation, but clearly, it is preferable to use a prediction based on synthetic promoters, as opposed to a native promoter that does not address this uncertainty.

To prove the stability predictor's accuracy, the inventors ran the herein disclosed sTAUbility Enhancer software with GFP as a target gene which ranked all of the yeast's ORFs according to their predicted co-stability. The inventors then tested whether there was a correlation between the predicted ranking and the empirical ranking of the nine validated genes analyzed in the herein disclosed proof-of-concept experiment. The dot-plot presented in FIG. 6 reflects the findings, showing that there was a significant correlation between the software's prediction and the empirical results. This result demonstrates the herein disclosed software's validity and accuracy in predicting which essential gene is most capable of prolonging a target gene's evolutionary half-life.

Furthermore, this result shows that using the fluorescence SWAT database as a stability measure for the model's training is reasonable, since there was significant correlation between the empirical stability from the proof-of-concept evolution experiment and the fluorescence predictions.

### Example 3

### Linker Selection

The sTAUbility Enhancer software enables the user to choose which kind of linker to use—fusion or 2A linker—and displays the best 10 or 4 linkers accordingly. The 2A linkers are ranked according to protein expression level they provide, while the fusion linkers are scored considering the maintenance of the target and conjugated gene's folding.

2A linkers—There are four main 2A sequences. In order to obtain a desired ratio of protein expression, it is important to select the right 2A construct. The conventional way to rank the 2A peptides is from the most efficient P2A, followed by T2A, E2A and F2A (Kim et al., 2014).

Fusion linkers—The fusion linkers included in this study are inter-domain linker peptides of natural multi-domain proteins. Therefore, they provide an ample source of potential linkers for novel fusion proteins. These linkers provide the conformation, flexibility and stability needed for a protein's biological function in its natural environment (George and Heringa, 2002).

The inventors devised a linker selection model, defining an ideal linker as one that minimally effects the essential and target proteins' natural 3D folding (which is assumed to be reflected by the disorder profile). The disordered nature of a protein segment can be context dependent: certain protein regions can switch between an ordered and a disordered state depending on various environmental factors. The IUPred2A tool (Mészáros et al., 2018) used in this study can detect such context-dependent disorder in the case where the environmental factors are either a change in the redox state or the presence of an ordered binding partner. Other 3D folding/disorder profile tools such as PONDR (Romero et al., 1997), DisEMBL (Linding et al., 2003), MoreRONN (Ramraj, 2014), ESpritz (Walsh et al., 2012), Foldlndex (Prilusky et al., 2005), etc. were tested for this purpose. However, the desired tool had to return a score for the folding's level that can be analyzed automatically with a short running time. Therefore, only IUPred2A was found suitable for the model.

To determine the score for each linker, the disorder profiles before and after the fusion were modeled for GFP and various yeast's ORFs. It was noticed that usually the score change occurs at a limited number of amino acids, the ones that are the closest to the linker. Therefore, the scores of the target and the essential genes (see Methods) were not normalized by their length. Moreover, most of the linkers had very low disorder score, meaning that they were naturally ordered. For this reason, the tendency of the linker to fold individually was not considered.

### Example 4

### Evolutionary Stability Optimizer—Novel Approach to Identify and Avoid Mutational Hotspots in DNA Sequences while Inducing High Expression Levels

### Introduction

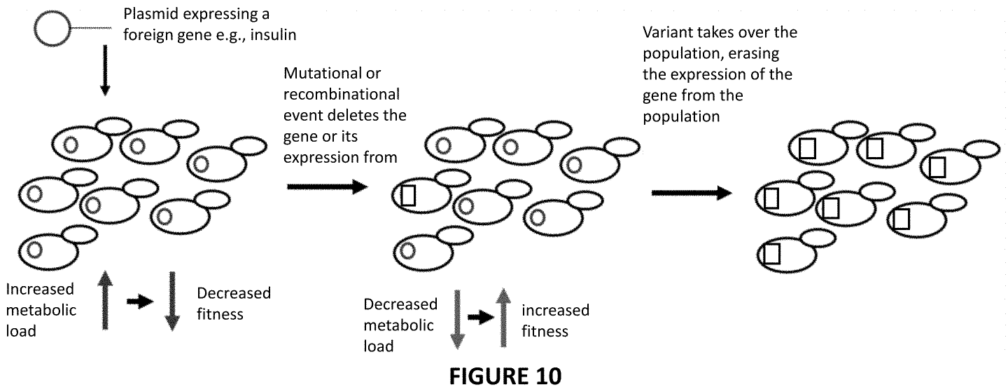

Recent advances in the quickly evolving field of synthetic biology have led to the development of various genetic circuits for therapeutics and bioproduction applications. However, once such a construct is inserted into a host organism, it imposes an additional burden on the host, because of (a) the metabolic load of constructing unrequired RNAs and proteins, and (b) heterologous genetic parts that interfere with native cellular processes. Both phenomena significantly reduce host fitness, leading to the presence of strong selective pressure against the genetic circuit. Therefore, loss-of-function mutations that damage the construct are likely to be selected for, diminishing or abolishing altogether the activity of the circuit. Because of their increased fitness, the mutated individuals will eventually take over the population (FIG. 10). These mutations could render synthetic-biology related products obsolete and require constant maintenance. Moreover, circuits with high evolutionary stability are known to have low expression levels. Thus, designing a DNA sequence specifically to withstand evolutionary failure while preserving or increasing expression levels is an important goal for synthetic biology.

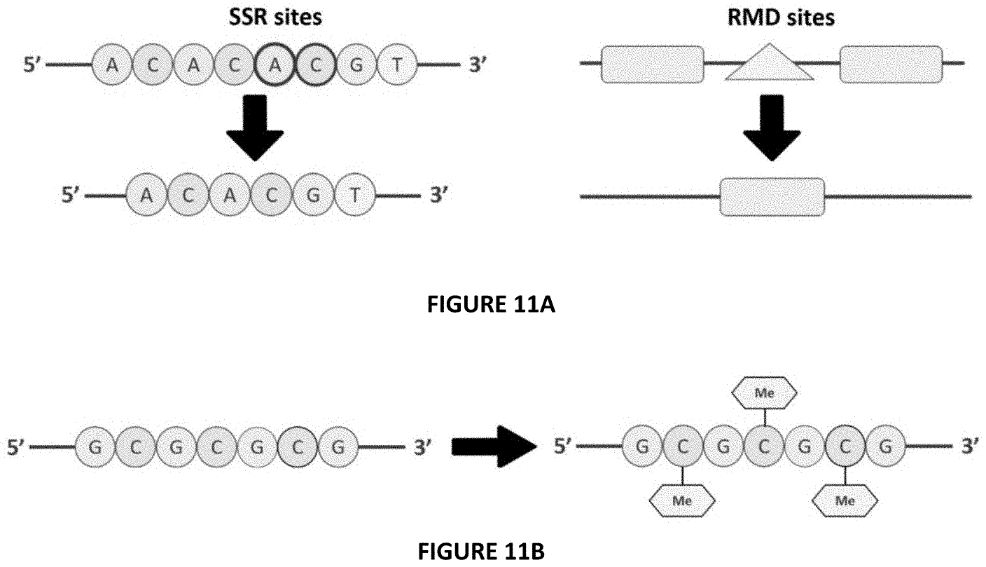

Generally, a small number of mutational hotspots in a certain construct are responsible for most of the mutations accumulated in that construct (FIG. 11). Their presence can destabilize any genetic circuit in nearly any organism. Some examples are: simple sequence repeats (SSR), sequences rich with repeating simple elements, that pose a challenge to replicative polymerases; and repeat mediated deletions (RMD), deletion events rising from unwanted recombination between long repeated sequences (11A). Another type of genetic instability that can befall on a certain construct is epigenetic changes in the expression patterns of the genes involved in the construct. Specifically, addition of a methyl group to adenine- or cytosine-containing sites (FIG. 11B) is known to repress inserted genes in insectoid and mammalian host cells. In addition, for unique, custom needs, users may provide their own sites to avoid through use of custom PSSM matrices.

These instability hotspots, if detected in advance, can be removed manually when planning a synthetic-biology construct. However, the creation of generic tools for the improvement of mutational stability is a surprisingly neglected field. One of the most known web tools that assist in such analysis is the Evolutionary Failure Mode (EFM) calculator [20], which enables prediction of potential mutational vulnerabilities in an input DNA sequence. Using empirical data collected from various studies, the calculator predicts the probability of mutation in the hypermutable sites of SSR and RMD and compares them with the Base Pair Substitution (BPS) rate (see Methods). Thus, high scoring sites within the genetic sequence are far more likely to mutate and can be erased or modified for a significant increase in evolutionary stability.

Recently, another tool called Nonrepetitive Parts Calculator (NRPC) has been presented by Cardenas, based on the machine learning and graph theoretic algorithms. In this work, given a maximal allowed length of repeating sequences between different parts, thousands of biological parts are generated and analyzed. This permits easy design of synthetic sequences, while avoiding RMD sites and significantly reducing the likelihood of mutation.

However, these tools are unsuited for the current direction of genetic work which becomes more systematic and large-scale, since they both perform single sequence analysis. Furthermore, neither addresses all possible mutation types, as the EFM calculator is unable to predict areas of epigenetic instability, and the NRPC refers only to RMD sites. Most importantly, their design principles do not consider the required trade-off between the contradictory demands of evolutionary stability and high expression level.

The inventors of the present invention disclose a next generation of the EFM calculator, termed “Evolutionary stability optimizer (ESO)”, a robust tool for automatic optimization of large-scale sequences for optimal genetic and epigenetic stability. This tool provides an end-to-end solution to the designing of stable constructs: it enables large-scale detection of SSR, RMD, methylation, or custom sites in multiple sequences at once, and offers optimization of these sequences with respect to both expression levels and genetic stability.

### Results and Discussion

The ESO features—A tool termed EFM Calculator was previously described (Jack et al., ACS Synth. Biol., 2014). The calculator finds and ranks SSR and RMD sites within a user's input sequence, allowing the users to manually delete or modify these sites as needed.

In an effort to create a more intuitive, flexible tool, that also enables DNA engineering and gene expression improvement, the inventors further modified the ESO. The herein disclosed modified ESO enables generation of stable, highly expressed genes to a much larger userbase, with much less invested time and effort. To reach these goals, the inventors included several important improvements on the detection mechanism provided by previous tool.

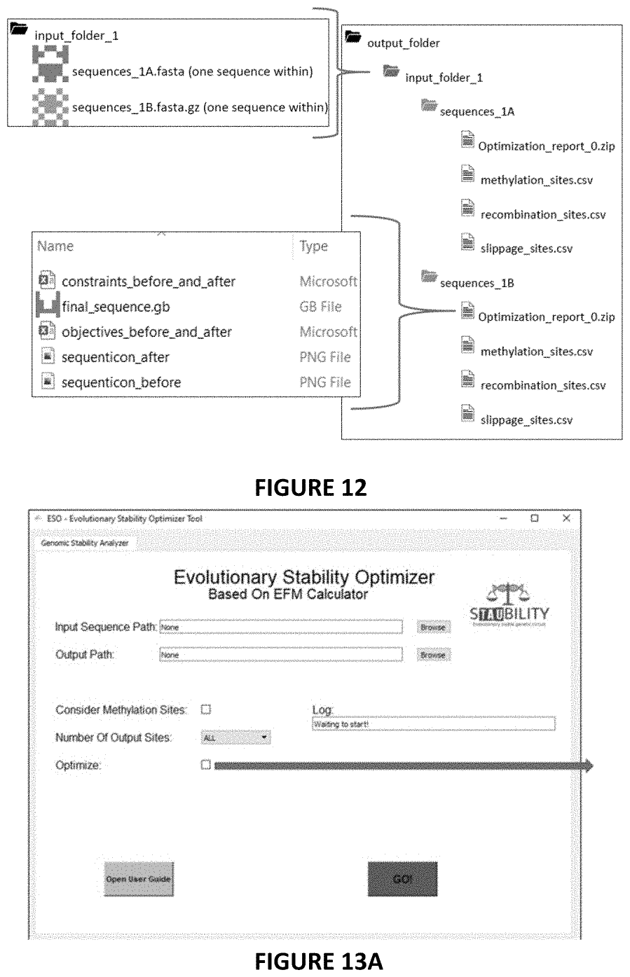

Large-scale analysis—Currently, the EFM calculator enables analysis of one sequence at a time, requiring manual insertion and exportation of the results. For larger projects with many sequences, this would be a significant bottleneck, leading to waste of time and possible file confusion. To address this issue within the software, the input is a directory, and all sequences within are analyzed. The results are placed within an output directory in a hierarchy-maintaining order, allowing the analysis of many sequences at once. Moreover, if the optimization option is being selected, an icon that is unique to each sequence is provided (https://github.com/Edinburgh-Genome-Foundry/sequenticon), allowing visual differentiation between sequences that otherwise might be confused with one another (FIG. 12).

Consideration of methylation sites—As previously discussed, mammalian and insectoid cells are much more sensitive to methylation sites than to SSR and RMD's. As such, any analysis that fails to take methylation into account, will return suboptimal results for these cells. Using the methylation detection mechanism (see Methods), the herein disclosed software finds and removes the sites most likely to match existing, known methylation sites.

Consideration of alternative sites—The methylation sites found are based on PSSM matrices provided by Wang et. al. The herein disclosed software is designed to be modular, providing support for updated or different requirements for optimization. Thus, users may provide their own PSSM matrices for sites to avoid, allowing much greater customizability for unique engineering needs.

Automatic optimization—The EFM calculator returns a list of hypermutable sites, with their location and ranking. This requires the user to invest much time and effort to manually correct the sequence, often reaching sub-optimal results. In the herein disclosed software, the inventors designed an optimization engine which avoids the identified hotspots, regulates the GC content, and increases the frequency of optimal codons. In addition to hotspot detection, the users are provided also with a final, ready-to-use sequence, optimized for stability and expression. Thus, the ESO provides an end-to-end solution, a concept that is yet to exist in the field of genomic stability analysis.

For any given input sequence, the optimization procedure involves two steps: (a) optimize codon usage and required GC content; (b) avoid mutational patterns (SSR, RMD and methylation when relevant) detected by the previous module in the semi-optimized sequence, while maintaining the codon usage and GC content as much as possible. This two-step strategy allows the algorithm to generate a sequence that is closer to optimum, and only then deal with mutational hotspots. Thus, the probability that new problematic sites will appear after optimization decreases dramatically.

The GC content optimization refers to the maintenance of the frequency of GC nucleotides within a specified range. The algorithm splits the sequence to windows of a specified size and optimize within each window. The user may choose to regulate the GC content according to the principles suitable for the host. For instance, in Saccharomyces cerevisiae, the lower the GC content, the more stable is the sequence, since it has been proved that genes with high GC had a substantially elevated rate of mutations, both single-base substitutions and deletions.

In codon optimization, the algorithm replaces the codons used to generate amino acids, in order to match the relative codon frequency within the host organism. The underlying assumption is that the genome of the host went through selective pressure for stability and expression in some form. Thus, by matching the sequence to the host, it will likely have higher levels of stability and expression as well. The optimization methods are “use best codon”, “match codon usage” and “harmonize RCA”, all described in the DNA chisel paper.

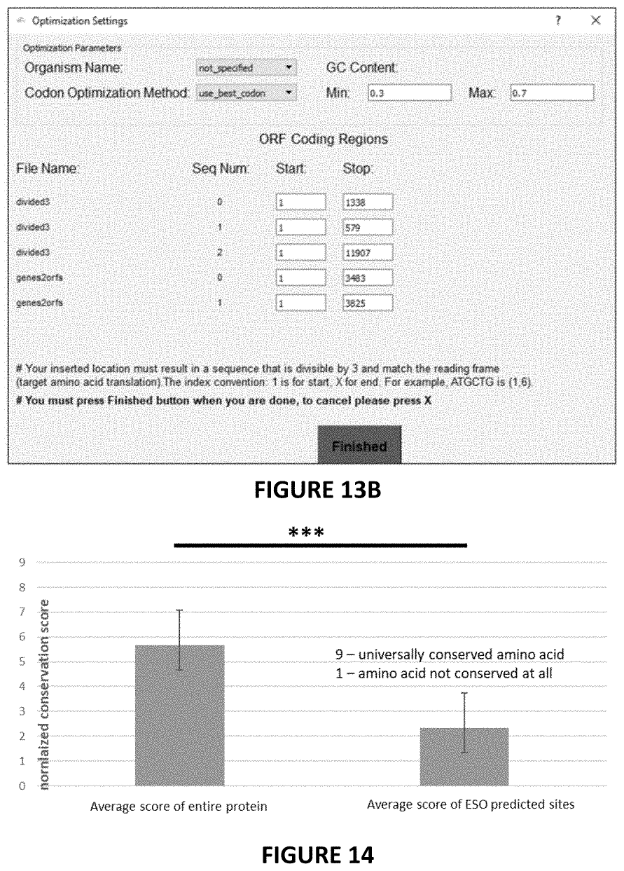

User interface—In order to provide an end-to-end solution and enable the above-mentioned analysis, the inventors developed a user-friendly software. The inventors wrapped this software in a Graphical User Interface (GUI, FIG. 13), downloadable as an application to the user's computer, thus allowing greater computational capabilities.

### The ESO Accurately Predicts the Evolutionary Stability of Endogenous Genes

To prove the efficiency and robustness of the herein disclosed modified ESO, the inventors analyzed the evolutionary stability of residues marked as unstable by the herein disclosed software. The inventors hypothesized that the areas marked by the ESO will have a lower conservation score, as they are genetically unstable.

For this analysis, the inventors selected 15 genes from Saccharomyces cerevisiae that are evolutionary conserved throughout the evolutionary tree. Genes were selected randomly from all genes in Saccharomyces cerevisiae that are conserved throughout all the eukaryotic realms. All 15 differed in cell localization, function, and length (from 271 aa to 1541 aa) and their list appears in the Methods section (see “Conservation Score Analysis”). The inventors then optimized these genes utilizing the ESO. The inventors calculated the average conservation score at each position for each selected gene and compared this value to: (1) The average of the lowest conservation score from each area predicted as unstable by our ESO's SSR calculator. (2) The average score from all the areas predicted by the RMD calculator (see Methods).

When addressing SSR, the inventors used only the lowest conserved nucleotide. The reason for this is because a mutation mediated by SSR will cause a single nucleotide level event (deletion, insertion of substation), while RMD mediated mutational event will influence all the predicted sites (large scale deletion of insertion).

The significance of the difference between the conservation average of the entirety of the gene and the areas marked by the ESO varied between 0.0016 in Pol30 to 0.0004 in Cdc9. In total, the inventors found that there was a significant difference between the conservation score of the entire gene and the conservation scores of the areas predicated by the ESO (whether it is SSR or RMD).

The results showed that the average normalized conservation score of areas in protein that were predicted to be evolutionary unstable is significantly lower than the rest of the protein (FIG. 14). Therefore, the areas chosen and then automatically modified by the ESO are indeed expected to be less conserved in evolution, compared to the rest of the protein. This suggests that the ESO software successfully predicts areas that are evolutionary unstable and automatically offers a new, optimized sequence with enhanced evolutionary stability.

### CONCLUSION

As the synthetic biology field evolves, the need for generic tools enabling the design of stable genetic constructs is rapidly increasing. The herein disclosed ESO software tool in its current state outperforms the various tools in the field by several aspects. Combining mutational hotspot as RMD and SSR with epigenetic hotspots prediction in one tool further widening the scope of utilization to eukaryotic organisms while allowing large scale analyzing not only for one given sequence. Custom site avoidance provides a solution for custom engineering needs. In addition, automatically optimizing the sequences for matching GC content and codon usage for given organism, while avoiding the mutational hotspots, increases stability and expression levels. The solutions are presented in a simple and attractive user interface.

The benefits of using the herein disclosed software are reflected not only in saving time but also in lowering costs of DNA designs. Optimized sequences prevent human error and are more likely to succeed, hence reducing the chance repeating the process. In addition, using the software can help a single end user in research as well as biotechnology companies in developing new products.

### Example 5

### Gene-SEQ (Stability Enhancing Quantifier)

The two main goals of this experiment are: (1) Ranking all the ORF in their ability to prolong the evolutionary half-life of a given gene; and (2) Gathering data on the mutational footprint of each ORF+given gene construct.

This mass amount of data can then be further analysed by the herein disclosed algorithm. This data, after processing, will first help determining the best match between the given gene used in the experiment and a specific ORF, and secondly, will help the herein disclosed artificial intelligence (AI) algorithm to be able to better accurately predict in the future the best match between every given gene and its best matching ORF.

The first step of the experiment is preparing a genetic library, in which the gene in question is fused to the N′ terminus of all the ORF (or to a smaller subset of ORF if chosen so) in the organism in question (whether it is yeast, bacteria, e.g., E. coli, or any other organism; meaning the experiment practically remains the same). Second step includes growing all the different strains of the library together as a co-culture in an evolution experiment for a period of time to the end user's choosing. The third and last step is harvesting the cells and preforming the Gene-SEQ protocol. The herein disclosed method utilises Nano-pore sequencing and RCA (rolling circle amplification). Nano-pore sequencing allows the sequencing of long reads of DNA, and thus, allows knowing how many mutations every ORF has accumulated, and what type of mutation. This, together with the data of how many reads were obtained from every ORF (meaning what was its fitness compared to the rest of the population) can help in determining every ORF fitness, ranking in prolonging genetic stability and mutational footprint. Such data is not obtained from Illumina sequencing. Because Nano-pore is an error-some sequencing (compared to Illumina), the inventors are preforming an RCA reaction, creating concatemers of repeats of every construct, thus allowing to differentiate between mutation in the construct and mutation in the sequencing process.

The gene-SEQ protocol uses restriction enzymes computationally chosen so they do not cut in the construct of the gene+ORF but also do not create a fragment too long for PCR reaction. Then using T4 ligase, those constructs are ligated, thus creating circular DNA which is then amplified using primers directed outward (accordingly a reaction accrues only in properly restricted and ligated constructs). The PCR product is then re-ligated and undergoes RCA reaction which creates concatemers as templates for the nano-pore sequencing. This data is then bioinformatically analyzed.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A composition comprising a plurality of transgenic cells comprising a polynucleotide encoding a chimeric polypeptide, the polynucleotide comprising at least a first nucleic acid sequence encoding a polypeptide of interest of said chimeric polypeptide and at least a second nucleic acid sequence encoding an essential protein of said chimeric polypeptide, and wherein at least 10% of said plurality of transgenic cells are genetically optimized such that the expression of said polypeptide of interest is substantially maintained after a period of at least 80 generations.

2. The composition of claim 1, wherein said at least first nucleic acid sequence encoding said polypeptide of interest is genetically optimized such that is comprises: a modified GC content, at least one less mutation hotspot, an optimized codon usage according to the preference of said transgenic cells, at least one less epigenetic hotspot, or any combination thereof, compared to a wildtype nucleic acid sequence encoding said polypeptide of interest, optionally wherein said at least one mutation hotspot comprises a simple sequence repeat (SSR), a repeated mediated deletion (RMD), or both, and optionally wherein said epigenetic hotspot comprises a methylation site.

3. (canceled)

4. (canceled)

5. The composition of claim 1, wherein any one of: (i) said substantially maintained comprises an expression of said polypeptide of interest after a period of at least 80 generations being at least 75% of the expression level of said polypeptide of interest after 1 generation; (ii) wherein at least 20% of said plurality of transgenic cells are genetically optimized such that the expression of said polypeptide of interest after a period of at least 80 generations is at least 75% of the expression level of said polypeptide of interest after 1 generation; (iii) wherein said transgenic cells are solitary cells; (iv) wherein said chimeric polypeptide comprises said polypeptide of interest N-terminally to said essential protein of said transgenic cells; (v) wherein said polypeptide of interest and said essential protein are not the same protein; (vi) wherein said essential protein is essential for: cell vitality, cell mitosis, cell metabolism, cell differentiation, DNA polymerization, RNA transcription, protein translation, housekeeping activity, and any combination thereof, of any one of said plurality of transgenic cells or replication, packaging, host cell recognition, infection efficiency, or any combination thereof, of a virus so as to infect said plurality of transgenic cells; and any combination thereof.

6. (canceled)

7. (canceled)

8. (canceled)

9. (canceled)

10. (canceled)

11. The composition of claim 1, wherein any one of: (i) said essential protein is the complete protein or a fragment thereof, comprising an essential function; (ii) wherein removal of expression of said essential protein from said transgenic cells induces death of said transgenic cells, replication arrest of said transgenic cells, or both; (iii) wherein said chimeric polypeptide further comprises a linker sequence between said first amino acid sequence and said second amino acid sequence; and any combination thereof.

12. (canceled)

13. (canceled)

14. The composition of claim 11, wherein any one of: (i) said linker sequence comprises 2 to 50 amino acids; (ii) said linker is a flexible linker; (iii) said linker is concatenated to said first amino acid sequence of said chimeric polypeptide and to said second amino acid sequence of said chimeric polypeptide, thereby providing optimal folding of both said first amino acid sequence of said chimeric polypeptide and said second amino acid sequence of said chimeric polypeptide; (iv) said provides optimal folding comprises: reduces folding disturbance, increases spatial separation, restores folding, or any combination thereof, of said first amino acid sequence and said second amino acid sequence of said chimeric polypeptide; and any combination thereof.

15. (canceled)

16. (canceled)

17. (canceled)

18. The composition of claim 1, wherein said chimeric polypeptide further comprises a protein-localization sequence, and optionally wherein any one of: (i) said protein-localization sequence is operably linked to said polypeptide of interest, and optionally wherein said protein-localization sequence is upstream of said first sequence; (ii) said localization is to a cellular location to which said essential protein localizes; (iii) said cellular location is selected from the group consisting of: nucleus, nucleolus, endoplasmic reticulum (ER), plasma membrane (PM), peroxisome, lysosome, centromere, centrosome, spindle, multivesicular bodies (MVBs), mitochondria, and exosome; and any combination thereof.

19. (canceled)

20. (canceled)

21. (canceled)

22. The composition of claim 1, wherein said chimeric polypeptide further comprises any one of a tag, a protease recognition site between said first amino acid sequence and said second amino acid sequence, and both.

23. (canceled)

24. The composition of claim 1, wherein any one of: (i) said transgenic cells are devoid of an endogenous functional form of said essential protein, optionally wherein any one of said transgenic cells are devoid of an endogenous essential protein; (ii) said transgenic cells comprise an endogenous genome being devoid of a gene encoding said functional essential protein, optionally wherein said endogenous genome is devoid of a gene encoding said essential protein; (iii) said transgenic cells are yeast cells, and any combination thereof.

25. (canceled)

26. (canceled)

27. A method for producing a polynucleotide molecule encoding a chimeric polypeptide comprising a polypeptide of interest, the method comprising:
a. generating or receiving a nucleic acid sequence comprising a coding region encoding a chimeric polypeptide, wherein said coding region comprises a 5′ region encoding said polypeptide of interest and a 3′ region encoding an essential gene of a target cell, optionally wherein said coding region comprises a region between the 5′ region and the 3′ region encoding a linker;
b. expressing said nucleic acid sequence in said target cell under conditions sufficient for expression of said chimeric polypeptide, wherein said target cell is devoid of an endogenous functional form of said essential protein;
c. culturing said target cell expressing said nucleic acid sequence for a time sufficient to determine if said chimeric protein can replace an essential function of said endogenous functional form of said essential protein; and
d. selecting said nucleic acid sequence if said chimeric polypeptide can replace said essential function;
thereby producing a polynucleotide molecule encoding a chimeric polypeptide.

28. The method of claim 27, wherein replacing an essential function of said endogenous functional form of said essential protein is replacing all essential functions of said endogenous functional form of said essential protein.

29. The method of claim 27, wherein said determining comprises determining if said cell dies, enters replication arrest, or both.

30. The method of claim 27, further comprises culturing said target cell expressing said nucleic acid sequence for a time sufficient to determine if said cell can lose expression of said protein of interest and still retain said essential function, and not selecting said polynucleotide molecule if said expression can be lost while retaining said essential function.

31. The method of claim 27, wherein said expressing comprises transferring an expression vector comprising said nucleic acid sequence into said target cell; or modifying a genome of said target cell to include said nucleic acid sequence.

32. The method of claim 27, wherein the method is a method of producing a polynucleotide molecule encoding a polypeptide of interest with increased genetic stability, optionally wherein said increased genetic stability is as compared to a polynucleotide molecule encoding a polypeptide of interest unlinked to said essential protein.

33. (canceled)

34. A method for genetically optimizing the expression of a polypeptide of interest such that it is substantially maintained in least 10% of a plurality of transgenic cells after a period of at least 80 generations of said cells, the method comprising:
a. receiving a nucleic acid sequence comprising a coding region encoding a polypeptide of interest;
b. generating a coding sequence encoding a chimeric polypeptide comprising the polypeptide of interest and an essential gene of a target cell optimized thereto for the generation of a chimeric polypeptide in the target cell, wherein said optimized comprises: a modified GC content, at least one less mutation hotspot, an modified codon usage for optimized expression of said coding sequence in said target cell, at least one less epigenetic hotspot, or any combination thereof, compared to a wildtype nucleic acid sequence encoding any one of: said polypeptide of interest, said essential gene of said target cell, and both; and wherein said chimeric polypeptide retains an essential function of said essential gene in said target cell; and
c. expressing said chimeric polypeptide in said plurality of transgenic cells.

35. The method of claim 34, wherein said selecting further comprises selecting a sequence encoding a linker being concatenated to said polypeptide and to said essential gene of said target cell, of said chimeric polypeptide, optionally wherein said selected linker provides optimal folding of both said polypeptide and said essential gene of said target cell, of said chimeric polypeptide.

36. (canceled)

37. The method of claim 35, wherein said selected linker: reduces folding disturbance, increases spatial separation, restores folding, or any combination thereof, of said polypeptide and said essential gene of said target cell, of said chimeric polypeptide.

38. The method of claim 34, wherein said at least one mutation hotspot comprises a simple sequence repeat (SSR), a repeated mediated deletion (RMD), or both.

39. The method of claim 34, wherein said epigenetic hotspot comprises a methylation site.

40. The method of claim 34, further comprising a step proceeding step (c), comprising determining the expression level of said polypeptide of interest of said chimeric polypeptide in said plurality of transgenic cells.
